(19) United States
(12) Patent Application Publication
(10) Pub. No.: US 2022/0119837 A1
(43) Pub. Date: Apr. 21, 2022

(54) GENE THERAPY DNA VECTOR BASED ON GENE THERAPY DNA VECTOR VTVAF17

(71) Applicants: CELL AND GENE THERAPY LTD, London (GB); OBSCHESTVO S OGRANICHEN-NOI OTVETSTVENNOSTJU "PRORYVNYE IN-NOVATSIONNYE TEKHNOLOGII", Moskva (RU)

(72) Inventor: Natalia SAVELIEVA, Wienna (AT)

(21) Appl. No.: 17/289,547

(22) PCT Filed: Jun. 10, 2019
(86) PCT No.: PCT/RU2019/000412

§ 371 Date: Apr. 28, 2021

(30) Foreign Application Priority Data
Jun. 8, 2018 (RU) 2018121339

(51) Int. Cl. C12N 15/85

(52) U.S. Cl. CPC C12N 15/85; A61K 48/00

(57) ABSTRACT

Gene-therapeutic DNA vectors based on the VTvaf17 gene-therapeutic DNA vector have been created for the treatment of diseases characterized by impaired mucociliary transport and mucolytic function and the development of mucostasis, including cystic fibrosis. The gene therapy DNA vector contains the coding region of the CFTR therapeutic gene, or the NOS1 therapeutic gene, or the AQ1 therapeutic gene, or the AQ3 therapeutic gene, or the AQ5 therapeutic gene. Methods for their preparation or use are proposed, as well as strains for the production of a gene therapy vector.

### FIELD OF THE INVENTION

The invention refers to genetic engineering and can be used in biotechnology, medicine, and agriculture for the manufacture of gene therapy products. I.e. the produced gene therapy DNA vector containing the therapeutic gene can be used to deliver it to the cells of human beings and animals that experience reduced or insufficient expression of that gene, thus ensuring the desired therapeutic effect.

### REFERENCE TO A SEQUENCE LISTING

SEQ ID NO: 1 through SEQ ID NO: 25, incorporated fully by reference herein, are provided in ASCII format together in one separately enclosed .TXT file, submitted via EFS-Web—File name: SEQ-LISTING412.txt; Date of Creation: Wednesday, May 26, 2021; File size: 38.6 KB.

### BACKGROUND OF THE INVENTION

Gene therapy is an innovative approach in medicine aimed at treating inherited and acquired diseases by means of delivery of new genetic material into a patient's cells to compensate for or suppress the function of a mutant gene and/or treat a genetic disorder.

Transporters of genetic material (gene therapy vectors) are divided into viral and non-viral vectors. Recently, gene therapy has paid increasingly more attention to the development of non-viral gene delivery systems with plasmid vectors topping the list.

Plasmid vectors are free of limitations inherent in viral vectors. In the target cell, they exist as an episome without being integrated into the genome, while producing them is quite cheap, and there is no immune response or side effects caused by the administration of plasmid vectors, which makes them a convenient tool for gene therapy and prevention of the genetic diseases (DNA vaccination) (Li L, Petrovsky N. Molecular mechanisms for enhanced DNA vaccine immunogenicity. Expert Rev Vaccines. 2016; 15(3):313-29).

However, limitations of plasmid vectors use in gene therapy are: 1) presence of antibiotic resistance genes for the production of constructs in carrying strains; 2) the presence of various regulatory elements represented by sequences of viral genomes; 3) size of therapeutic plasmid vector that determines the efficiency of vector delivery to the target cell.

It is known that the European Medicines Agency deems it necessary to refrain from adding antibiotic resistance marker genes to newly engineered plasmid vectors for gene therapy (Reflection paper on design modifications of gene therapy medicinal products during development/14 Dec. 2011 EMA/CAT/GTWP/44236/2009 Committee for advanced therapies).

In addition, the same agency recommends avoiding the presence of regulatory elements in therapeutic plasmid vectors to increase the expression of therapeutic genes (promoters, enhancers, post-translational regulatory elements) that constitute nucleotide sequences of genomes of various viruses (Draft Guideline on the quality, non-clinical and clinical aspects of gene therapy medicinal products,

http://www.ema.europa.eu/docs/en_GB/document_library/Scientific_guideline/2015/05/WC50 0187020.pdf).

The size of the therapy vector is also essential. It is known that modern plasmid vectors often have unnecessary, non-functional sites that increase their length substantially (Mairhofer J, Grabherr R. Rational vector design for efficient non-viral gene delivery: challenges facing the use of plasmid DNA. Mol Biotechnol. 2008.39(2):97-104).

A method has been known for accumulating plasmid vectors in Escherichia coli strains without using antibiotics (Cranenburgh R M, Hanak J A, Williams S G, Sherratt D J. Escherichia coli strains that allow antibiotic-free plasmid selection and maintenance by repressor titration. Nucleic Acids Res. 2001. 29(5):E26). DH1lacdapD and DH1lacP2dapD strains of Escherichia coli were constructed, where gene dapD encoding 2,3,4,5-tetrahydropyridine-2,6-dicarboxylate-N-succinyltransferase enzyme involved in the biosynthesis of L-lysine is controlled by the lac promoter. In the absence of the inducer IPTG (Isopropyl-β-D-1-thiogalactopyranoside), these strains are subject to lysis. However, the administration of the pORT multicopy plasmid vector containing the lac operon induces expression of gene dapD, and, therefore, transformed clones may be selected and reproduced. These strains, however, feature low levels and instability of transformation.

An invention is reported in U.S. Patent Application No. 2011152377/10 for the preparation of an expression plasmid vector without the resistance to antibiotics that contains a polynucleotide sequence encoding the repressor protein. The expression of the said repressor protein regulates the expression of the toxic gene product integrated into the region of the E. coli genome. However, like any other method of selection based on the use of repressor proteins, this method features unstable and inefficient transformation.

U.S. Pat. No. 9,644,211 describes a method for producing a vector of the smallest length. This vector does not contain bacterial genome sequences and is produced by parA-mediated recombination in a cultured E. coli strain. The disadvantage of this method of producing the shortest vector is the impossibility to use it on an industrial scale.

The prototype of this invention in terms of the use of recombinant DNA vectors for gene therapy is the method of producing a recombinant vector for genetic immunisation (U.S. Pat. No. 9,550,998). The plasmid vector is a supercoiled plasmid DNA vector that is used for the expression of cloned genes in human and animal cells. The vector contains an origin of replication, regulatory elements comprising human cytomegalovirus promoter and enhancer, and regulatory sequences from the human T-cell lymphotropic virus.

The vector is accumulated in a dedicated E. coli strain free of antibiotics through antisense complementation of sacB gene administered into the strain by means of bacteriophage. The use of this DNA vector in gene therapy is limited by the presence of regulatory sequences of viral genomes.

### DISCLOSURE OF THE INVENTION

The purpose of the invention is to construct gene therapy DNA vectors for the treatment of diseases associated with the need to increase the expression level of these therapeutic genes that would reasonably combine the following:

I) possibility of safe use in the gene therapy of human beings and animals due to the absence of antibiotic resistance genes in the gene therapy DNA vector,

II) length that ensures efficient gene delivery to the target cell,

III) presence of regulatory elements that ensure efficient expression of the therapeutic genes while not being represented by nucleotide sequences of viral genomes,

IV) producibility and constructability on an industrial scale.

Item I and III are critical and are provided herein in compliance with the requirements of the state regulators for gene therapy medicines and, specifically, the requirement of the European Medicines Agency to refrain from adding antibiotic resistance marker genes to newly engineered plasmid vectors for gene therapy (Reflection paper on design modifications of gene therapy medicinal products during development/14 Dec. 2011 EMA/CAT/GTWP/44236/2009 Committee for advanced therapies) and refrain from adding viral genomes to newly engineered plasmid vectors for gene therapy (Guideline on the quality, non-clinical and clinical aspects of gene therapy medicinal products/23 Mar. 2015, EMA/CAT/80183/2014, Committee for Advanced Therapies).

The purpose of the invention also includes the construction of strains carrying these gene therapy DNA vectors for the production of these gene therapy DNA vectors on an industrial scale.

The specified purpose is achieved by using the produced gene therapy DNA vectors based on the gene therapy DNA vector VTvaf17 for the treatment of diseases featuring disruption of mucociliary transport and mucolytic function and development of mucostasis, including cystic fibrosis, while the gene therapy DNA vector contains the coding region of the CFTR therapeutic gene cloned to gene therapy DNA vector VTvaf17 to produce a 7606 bp gene therapy DNA vector VTvaf17-CFTR featuring nucleotide sequence SEQ ID No. 1; the gene therapy DNA vector contains the coding region of the NOS1 therapeutic gene cloned to gene therapy DNA vector VTvaf17 to produce a 7468 bp gene therapy DNA vector VTvaf17-NOS1 featuring nucleotide sequence SEQ ID No. 2; the gene therapy DNA vector contains the coding region of the AQ1 therapeutic gene cloned to gene therapy DNA vector VTvaf17 to produce a 3982 bp gene therapy DNA vector VTvaf17-AQ1 featuring nucleotide sequence SEQ ID No. 3; the gene therapy DNA vector contains the coding region of the AQ3 therapeutic gene cloned to gene therapy DNA vector VTvaf17 to produce a 4024 bp gene therapy DNA vector VTvaf17-AQ3 featuring nucleotide sequence SEQ ID No. 4; the gene therapy DNA vector contains the coding region of the AQ5 therapeutic gene cloned to gene therapy DNA vector VTvaf17 to produce a 3943 bp gene therapy DNA vector VTvaf17-AQ5 featuring nucleotide sequence SEQ ID No. 5.

The method of producing a gene therapy DNA vector based on gene therapy DNA vector VTvaf17 carrying CFTR, or NOS1, or AQ1, or AQ3, or AQ5 therapeutic gene that involves obtaining each of the gene therapy DNA vectors: DNA vector VTvaf17-CFTR, or VTvaf17-NOS1, or VTvaf17-AQ1, or VTvaf17-AQ3, or VTvaf17-AQ5 as follows: the coding region of CFTR, or NOS1, or AQ1, or AQ3, or AQ5 therapeutic genes is cloned to gene therapy DNA vector VTvaf17, and gene therapy DNA vector VTvaf17-CFTR, SEQ ID No. 1, or VTvaf17-NOS1, SEQ ID No. 2, or VTvaf17-AQ1, SEQ ID No. 3, or VTvaf17-AQ3, SEQ ID No. 4, or VTvaf17-AQ5, SEQ ID No. 5 is obtained, respectively, while the coding region of the therapeutic gene CFTR, or NOS1, or AQ1, or AQ3, or AQ5 is obtained by isolating the total RNA from a biological sample of human tissues followed by the reverse transcription reaction and PCR amplification using the produced oligonucleotides and cleavage of the amplification product by corresponding restriction endonucleases, and cloning to gene therapy DNA vector VTvaf17 performed by SalI and KpnI restriction sites, or BamHI and EcoRI restriction sites, and the selection is made without antibiotics,

and the following oligonucleotides produced for this purpose are used during gene therapy DNA vector VTvaf17-CFTR, SEQ ID No. 1 production for the reverse transcription reaction and PCR amplification:

CFTR_F
ATCGTCGACCGCCATGCAGAGGTCGCCT,
CFTR_R
TGGTACCTTAAAGCCTTGTATCTTGCACCTC,

and cleavage of the amplification product and cloning of the coding region of CFTR gene to gene therapy DNA vector VTvaf17 is carried out using Sall and Kpnl restriction endonucleases,

and the following oligonucleotides produced for this purpose are used during gene therapy DNA vector VTvaf17-NOS1, SEQ ID No. 2 production for the reverse transcription reaction and PCR amplification:

NOS1_F
ATCGTCGACTACCATGGAGGATCACATG,
NOS1_R
CGGTACCTTAGGAGCTGAAAACCTCATC,

and cleavage of the amplification product and cloning of the coding region of NOS1 gene to gene therapy DNA vector VTvaf17 is carried out using Sall and Kpnl restriction endonucleases,

and the following oligonucleotides produced for this purpose are used during gene therapy DNA vector VTvaf17-AQ1, SEQ ID No. 3 production for the reverse transcription reaction and PCR amplification:

AQ1_F
TGGATCCAGCGGTCTCAGGCCAAG,
AQ1_R
CCAGAATTCTTCTATTTGGGCTTCATCTC,

and cleavage of the amplification product and cloning of the coding region of AQ1 gene to gene therapy DNA vector VTvaf17 is carried out using BamHI and EcoRI restriction endonucleases,

and the following oligonucleotides produced for this purpose are used during gene therapy DNA vector VTvaf17-AQ3, SEQ ID No. 4 production for the reverse transcription reaction and PCR amplification:

AQ3_F
TGGATCCCGCCATGGGTCGACAG,
AQ3_R
TCTGAATTCTCAGATCTGCTCCTTGTGCT,

and cleavage of the amplification product and cloning of the coding region of AQ3 gene to gene therapy DNA vector VTvaf17 is carried out using BamHI and EcoRl restriction endonucleases,

and the following oligonucleotides produced for this purpose are used during gene therapy DNA vector VTvaf17-AQ5, SEQ ID No. 5 production for the reverse transcription reaction and PCR amplification:

AQ5_F
AGGATCCCACCATGAAGAAGGAGGTG,
AQ5_R
GGAATTCTCAGCGGGTGGTCAGCTCC,

and cleavage of the amplification product and cloning of the coding region of AQ5 gene in gene therapy DNA vector VTvaf17 is carried out using SalI and HindIII restriction endonucleases.

A method of use of gene therapy DNA vector based on gene therapy DNA vector VTvaf17 carrying CFTR, or NOS1, or AQ1, or AQ3, or AQ5 therapeutic gene for the treatment of diseases featuring disruption of mucociliary transport and mucolytic function and development of mucostasis, including cystic fibrosis, that involves transfection with the selected gene therapy DNA vector carrying the therapeutic gene based on gene therapy DNA vector VTvaf17, or several selected gene therapy DNA vectors carrying therapeutic genes based on gene therapy DNA vector VTvaf17, from the constructed gene therapy DNA vectors carrying therapeutic genes based on gene therapy DNA vector VTvaf17, of the cells of patient or animal organs and tissues, and/or the injection of autologous cells of patient or animal into the organs and tissues of the same patient or animal transfected by the selected gene therapy DNA vector carrying therapeutic gene based on gene therapy DNA vector VTvaf17 or several selected gene therapy DNA vectors carrying the therapeutic genes based on gene therapy DNA vector VTvaf17 from the group of constructed gene therapy DNA vectors carrying therapeutic genes based on gene therapy DNA vector VTvaf17, and/or the injection of the patient or animal organs and tissues with the selected gene therapy DNA vector carrying therapeutic gene based on gene therapy DNA vector VTvaf17 or several selected gene therapy DNA vectors carrying therapeutic genes based on gene therapy DNA vector VTvaf17 from the group of constructed gene therapy DNA vectors carrying therapeutic genes based on gene therapy DNA vector VTvaf17, or the combination of the indicated methods.

The method of producing strain for production of gene therapy DNA vector for treatment of diseases featuring disruption of mucociliary transport and mucolytic function and development of mucostasis, including cystic fibrosis, and associated with the need to increase the expression level of the therapeutic genes that involves making electrocompetent cells of Escherichia coli strain SCS110-AF and subjecting these cells to electroporation with gene therapy DNA vector VTvaf17-CFTR, or DNA vector VTvaf17-NOS1, or DNA vector VTvaf17-AQ1, or DNA vector VTvaf17-AQ3, or DNA vector VTvaf17-AQ5, followed by pouring the cells into agar plates (Petri dishes) with a selective medium containing yeastrel, peptone, 6% sucrose, and 10 μg/ml of chloramphenicol, which results in obtaining Escherichia coli strain SCS110-AFNTvaf17-CFTR, or Escherichia coli strain SCS110-AF/VTvaf17-NOS1, or Escherichia coli strain SCS110-AF/VTvaf17-AQ1, or Escherichia coli strain SCS110-AF/VTvaf17-AQ3, or Escherichia coli strain SCS110-AF/VTvaf17-AQ5.

Escherichia coli strain SCS110-AF/VTvaf17-CFTR obtained by the method described above carrying gene therapy DNA vector VTvaf17-CFTR for its further production allowing for antibiotic-free selection during obtaining of gene therapy DNA vector for treatment of diseases featuring disruption of mucociliary transport and mucolytic function and development of mucostasis, including cystic fibrosis.

Escherichia coli strain SCS110-AF/VTvaf17-NOS1 obtained by the method described above carrying gene therapy DNA vector VTvaf17-NOS1 for its further production allowing for antibiotic-free selection during obtaining of gene therapy DNA vector for treatment of diseases featuring disruption of mucociliary transport and mucolytic function and development of mucostasis, including cystic fibrosis.

Escherichia coli strain SCS110-AF/VTvaf17-AQ1 obtained by the method described above carrying gene therapy DNA vector VTvaf17-AQ1 for its further production allowing for antibiotic-free selection during obtaining of gene therapy DNA vector for treatment of diseases featuring disruption of mucociliary transport and mucolytic function and development of mucostasis, including cystic fibrosis.

Escherichia coli strain SCS110-AF/VTvaf17-AQ3 obtained by the method described above carrying gene therapy DNA vector VTvaf17-AQ3 for its further production allowing for antibiotic-free selection during obtaining of gene therapy DNA vector for treatment of diseases featuring disruption of mucociliary transport and mucolytic function and development of mucostasis, including cystic fibrosis.

Escherichia coli strain SCS110-AF/VTvaf17-AQ5 obtained by the method described above carrying gene therapy DNA vector VTvaf17-AQ5 for its further production allowing for antibiotic-free selection during obtaining of gene therapy DNA vector for treatment of diseases featuring disruption of mucociliary transport and mucolytic function and development of mucostasis, including cystic fibrosis.

The method of production gene therapy DNA vector based on gene therapy DNA vector VTvaf17 carrying CFTR, or NOS1, or AQ1, or AQ3, or AQ5 therapeutic gene on an industrial scale for treatment of diseases featuring disruption of mucociliary transport and mucolytic function and development of mucostasis, including cystic fibrosis that involves obtaining gene therapy DNA vector VTvaf17-CFTR, or gene therapy DNA vector VTvaf17-NOS1, or gene therapy DNA vector VTvaf17-AQ1, or gene therapy DNA vector VTvaf17-AQ3, or gene therapy DNA vector VTvaf17-AQ5 by inoculating the seed culture selected from Escherichia coli strain SCS110-AF/VTvaf17-CFTR, or Escherichia coli SCS110-AF/VTvaf17-NOS1, or Escherichia coli strain SCS110-AF/VTvaf17-AQ1, or Escherichia coli strain SCS110-AF/VTvaf17-AQ3, or Escherichia coli strain SCS110-AF/VTvaf17-AQ5 into a culture flask. Then the cell culture is incubated in an incubator shaker and transferred to an industrial fermenter, then grown to a stationary phase, then the fraction containing the therapeutic DNA product is extracted, multi-stage filtered, and purified by chromatographic methods.

The essence of the invention is explained in the drawings, where:

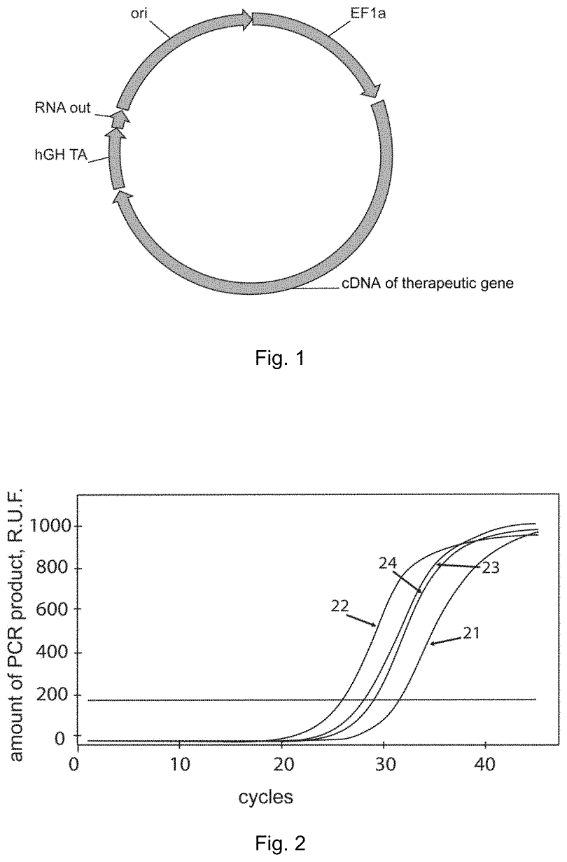

FIG. 1 shows the structure of gene therapy DNA vector VTvaf17 carrying cDNA of the therapeutic human gene selected from CFTR, or NOS1, or AQ1, or AQ3, or AQ5 that is a circular double-stranded DNA molecule capable of autonomous replication in Escherichia coli cells.

FIG. 1 marks the following structural elements of the vector:

(1) EF1a—the promoter region of human elongation factor EF1A with an intrinsic enhancer contained in the first intron of the gene. It ensures efficient transcription of the recombinant gene in most human tissues.
(2) cDNA of therapeutic gene,
(3) hGH-TA—the transcription terminator and the polyadenylation site of the human growth factor gene,
(4) RNA-out—the regulatory element RNA-OUT of transposon Tn 10 allowing for antibiotic-free positive selection in case of the use of Escherichia coli strain SCS 110-AF,
(5) ori—the origin of replication for autonomous replication with a single nucleotide substitution to increase plasmid production in the cells of most Escherichia coli strains.

FIG. 2 shows the patterns of accumulation of mRNA therapeutic gene, namely CFTR gene, in CFTE29o—human tracheal epithelial cell line before their transfection and 48 hours after transfection of these cells with DNA vector VTvaf17-CFTR carrying a region of human CFTR gene for the purpose of analysing changes in the accumulation of CFTR mRNA in tracheal epithelial cells before their transfection and 48 hours after transfection of these cells with DNA vector VTvaf17-CFTR carrying a region encoding CFTR gene, where:

21—cDNA of CFTR gene in human tracheal epithelial cell line CFTE29o—before transfection with DNA vector VTvaf17-CFTR,
22—cDNA of CFTR gene in human tracheal epithelial cell line CFTE29o—after transfection with DNA vector VTvaf17-CFTR,
23—cDNA of B2M gene in human tracheal epithelial cell line CFTE29o—before transfection with DNA vector VTvaf17-CFTR,
24—cDNA of B2M gene in human tracheal epithelial cell line CFTE29o—after transfection with DNA vector VTvaf17-CFTR.

B2M (beta-2-microglobuline) gene listed in the GenBank database under number NM 004048.2 was used as a reference gene.

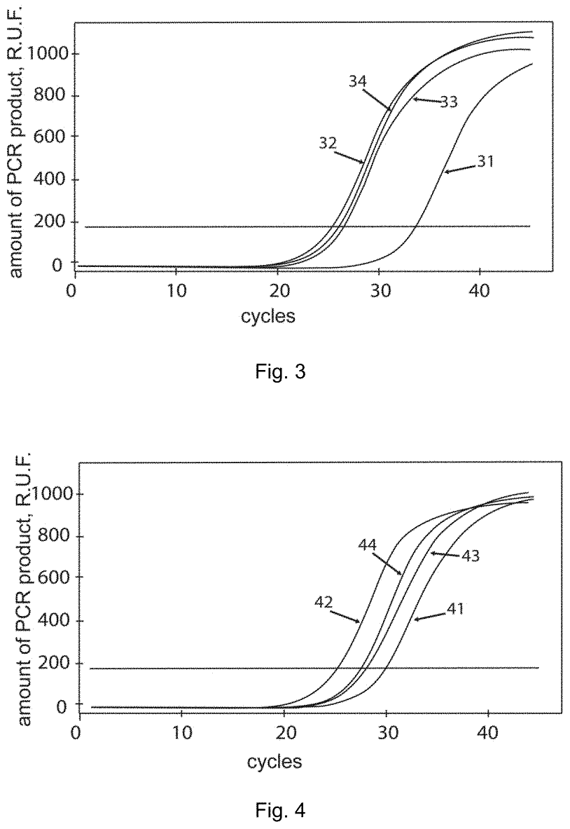

FIG. 3 shows the patterns of accumulation of mRNA therapeutic gene, namely NOS1 gene, in SH-SY5Y human neuroblastoma cells before their transfection and 48 hours after transfection of these cells with DNA vector VTvaf17-NOS1 carrying a region of human NOS1 gene for the purpose of analysing changes in the accumulation of NOS1 mRNA in SH-SY5Y cells before their transfection and 48 hours after transfection of these cells with DNA vector VTvaf17-NOS1 carrying a region encoding NOS1 gene, where:

31—cDNA of NOS1 gene in SH-SY5Y human neuroblastoma cells before transfection with DNA vector VTvaf17-NOS1,
32—cDNA of NOS1 gene in SH-SY5Y human neuroblastoma cells after transfection with DNA vector VTvaf17-NOS1,
33—cDNA of B2M gene in SH-SY5Y human neuroblastoma cells before transfection with DNA vector VTvaf17-NOS1,
34—cDNA of B2M gene in SH-SY5Y human neuroblastoma cells after transfection with DNA vector VTvaf17-NOS1.

B2M (beta-2-microglobuline) gene listed in the GenBank database under number NM 004048.2 was used as a reference gene.

FIG. 4 shows the patterns of accumulation of mRNA therapeutic gene, namely AQ1 gene, in human tracheal epithelial cell line CFTE29o—before their transfection and 48 hours after transfection of these cells with DNA vector VTvaf17-AQ1 carrying a region of human AQ1 gene for the purpose of analysing changes in the accumulation of AQ1 mRNA in CFTE29o—cells before their transfection and 48 hours after transfection of these cells with DNA vector VTvaf17-AQ1 carrying a region encoding AQ1 gene, where:

41—cDNA of AQ1 gene in human tracheal epithelial cell line CFTE29o—before transfection with DNA vector VTvaf17-AQ1,
42—cDNA of AQ1 gene in human tracheal epithelial cell line CFTE29o—after transfection with DNA vector VTvaf17-AQ1,
43—cDNA of B2M gene in human tracheal epithelial cell line CFTE29o—before transfection with DNA vector VTvaf17-AQ1,
44—cDNA of B2M gene in human tracheal epithelial cell line CFTE29o—after transfection with DNA vector VTvaf17-AQ1.

B2M (beta-2-microglobuline) gene listed in the GenBank database under number NM 004048.2 was used as a reference gene.

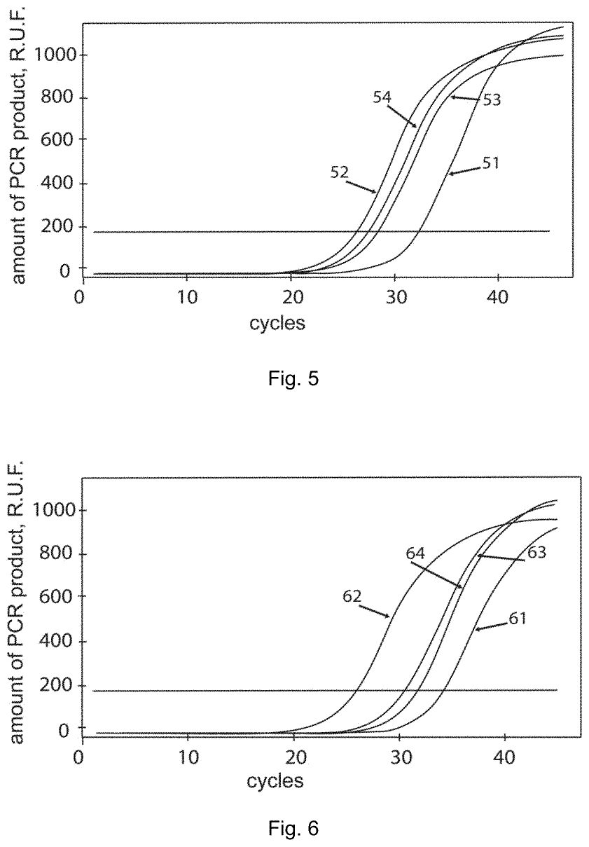

FIG. 5 shows the patterns of accumulation of mRNA therapeutic gene, namely AQ3 gene, in human primary small airway epithelial cells HSAECs before their transfection and 48 hours after transfection of these cells with DNA vector VTvaf17-AQ3 carrying a region of human AQ3 gene for the purpose of analysing changes in the accumulation of AQ3 mRNA in cells before their transfection and 48 hours after transfection of these cells with DNA vector VTvaf17-AQ3 carrying a region encoding AQ3 gene, where:

51—cDNA of AQ3 gene in human primary small airway epithelial cells before transfection with DNA vector VTvaf17-AQ3,
52—cDNA of AQ3 gene in human primary small airway epithelial cells after transfection with DNA vector VTvaf17-AQ3,
53—cDNA of gene in human primary small airway epithelial cells before transfection with DNA vector VTvaf17-AQ3,
54—cDNA of B2M gene in human primary small airway epithelial cells after transfection with DNA vector VTvaf17-AQ3.

B2M (beta-2-microglobuline) gene listed in the GenBank database under number NM 004048.2 was used as a reference gene.

FIG. 6 shows the patterns of accumulation of mRNA therapeutic gene, namely AQ5 gene, in human primary bladder smooth muscle cells HBdSMC (ATCC PCS-420-012) before their transfection and 48 hours after transfection of these cells with DNA vector VTvaf17-AQ5 carrying a region of human AQ5 gene for the purpose of analysing changes in the accumulation of AQ5 mRNA in HBdSMC cells before their transfection and 48 hours after transfection of these cells with DNA vector VTvaf17-AQ5 carrying a region encoding AQ5 gene, where:

61—cDNA of AQ5 gene in human urinary bladder smooth muscle cells before transfection with DNA vector VTvaf17-AQ5,
62—cDNA of AQ5 gene in human urinary bladder smooth muscle cells after transfection with DNA vector VTvaf17-AQ5,
63—cDNA of B2M gene in human urinary bladder smooth muscle cells before transfection with DNA vector VTvaf17-AQ5,
64—cDNA of B2M gene in human urinary bladder smooth muscle cells after transfection with DNA vector VTvaf17-AQ5.

B2M (beta-2-microglobuline) gene listed in the GenBank database under number NM 004048.2 was used as a reference gene.

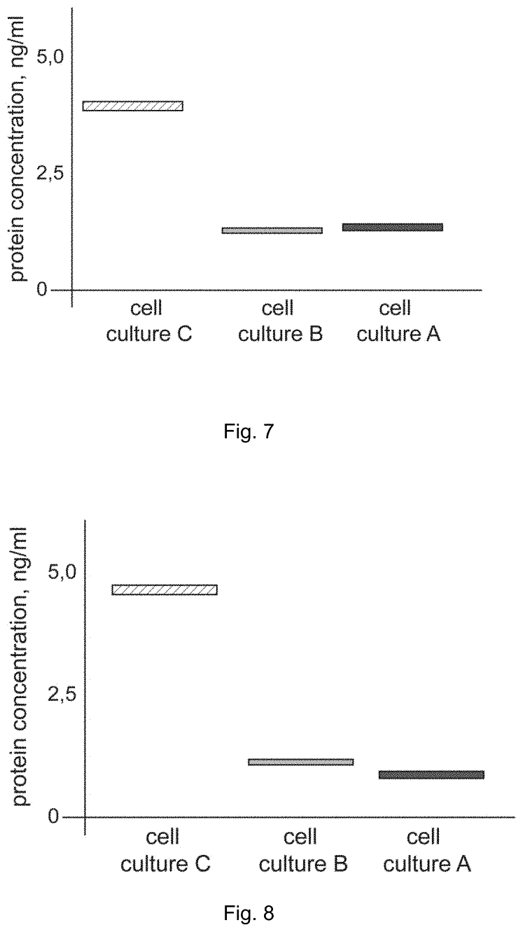

FIG. 7 shows the plot of protein concentration of transmembrane cystic fibrosis regulator protein in the cell lysate of CFTE29o—human tracheal epithelial cell line after transfection of these cells with DNA vector VTvaf17-CFTR carrying a region of human CFTR gene for the purpose of analysing changes in transmembrane cystic fibrosis regulator protein concentrations in the cell lysate of CFTE29o—human tracheal epithelial cell line upon transfection of these cells with DNA vector VTvaf17-CFTR carrying a region encoding CFTR gene, where:

culture A—culture of CFTE29o—human tracheal epithelial cell line transfected with aqueous dendrimer solution without DNA vector (reference),
culture B—culture of CFTE29o—human tracheal epithelial cell line transfected with DNA vector VTvaf17,
culture C—culture of CFTE29o—human tracheal epithelial cell line transfected with DNA vector VTvaf17-CFTR carrying a region of CFTR gene.

FIG. 8 shows the plot of protein concentration of neuronal nitric oxide synthase 1 in lysate of human neuroblastoma cells SH-SY5Y after transfection of these cells with the DNA vector VTvaf17-NOS1 carrying a region of human NOS1 gene for the purpose of analysing changes in protein concentrations of neuronal nitric oxide synthase 1 in lysate of human neuroblastoma cells SH-SY5Y upon transfection of these cells with DNA vector VTvaf17-NOS1 carrying a region encoding NOS1 gene, where:

culture A—SH-SY5Y cells transfected with aqueous dendrimer solution without plasmid DNA (reference),
culture B—SH-SY5Y cells transfected with DNA vector VTvaf17,
culture C—SH-SY5Y cells transfected with DNA vector VTvaf17-NOS1 carrying a region of NOS1 gene.

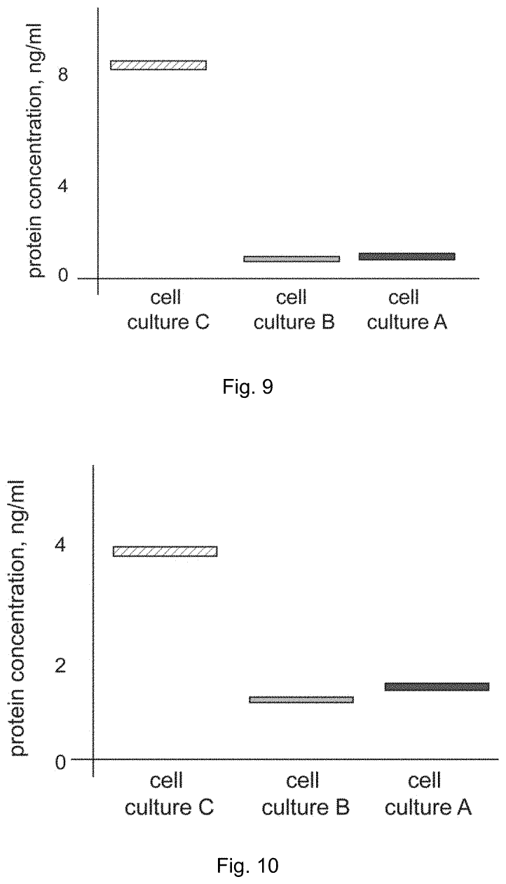

FIG. 9 shows the plot of aquaporin 1 protein concentration in the cell lysate of CFTE29o—human tracheal epithelial cell line after transfection of these cells with DNA vector VTvaf17-AQ1 carrying a region of human AQ1 gene for the purpose of analysing changes in aquaporin 1 protein concentrations in the human tracheal epithelial cell lysate upon transfection of these cells with DNA vector VTvaf17-AQ1 carrying a region encoding AQ1 gene, where:

culture A—culture of CFTE29o—human tracheal epithelial cell line transfected with aqueous dendrimer solution without DNA vector (reference),
culture B—culture of CFTE29o—human tracheal epithelial cell line transfected with DNA vector VTvaf17,
culture C—culture of CFTE29o—human tracheal epithelial cell line transfected with DNA vector VTvaf17-AQ1 carrying a region of AQ1 gene.

FIG. 10 shows the plot of aquaporins 5 protein concentration in the human primary bladder smooth muscle cell lysate HBdSMC after transfection of these cells with DNA vector VTvaf17- AQ5 carrying a region of human AQ5 gene for the purpose of analysing changes in aquaporin 5 protein concentration in the human primary bladder smooth muscle cell lysate HBdSMC upon transfection of these cells with DNA vector VTvaf17-AQ5 carrying a region encoding AQ5 gene, where:

culture A—human primary bladder smooth muscle cells transfected with aqueous dendrimer solution without DNA vector (reference),
culture B—human primary bladder smooth muscle cells transfected with DNA vector VTvaf17,
culture C—human primary bladder smooth muscle cells transfected with DNA vector VTvaf17-AQ5 carrying a region of AQ5 gene.

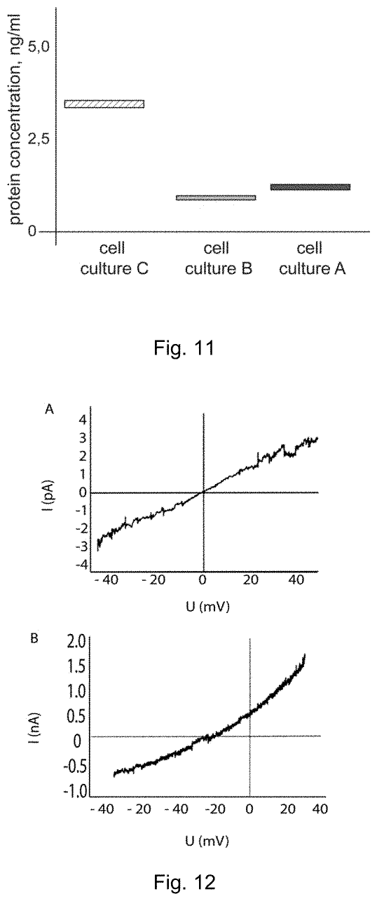

FIG. 11 shows the plot of aquaporin 3 protein concentration in cell lysate of culture of CFTE29o—human tracheal epithelial cell line after transfection of these cells with DNA vector VTvaf17-AQ3 carrying a region of human AQ3 gene for the purpose of analysing changes in aquaporin 3 protein concentrations in lysate of the culture of CFTE29o—human tracheal epithelial cell line upon transfection of these cells with DNA vector VTvaf17-AQ3 carrying a region encoding AQ3 gene, where:

culture A—culture of CFTE29o—human tracheal epithelial cell line transfected with aqueous dendrimer solution without DNA vector (reference),
culture B—culture of CFTE29o—human tracheal epithelial cell line transfected with DNA vector VTvaf17,
culture C—culture of CFTE29o—human tracheal epithelial cell line transfected with DNA vector VTvaf17-AQ3 carrying a region of AQ3 gene.

FIG. 12 shows volt-ampere characteristics of CFTE29o—(A) non-transfected cells and CFTE29o—cells transfected with DNA vector VTvaf17-CFTR, expressing CFTR gene (B) without Na+ and K+ currents.

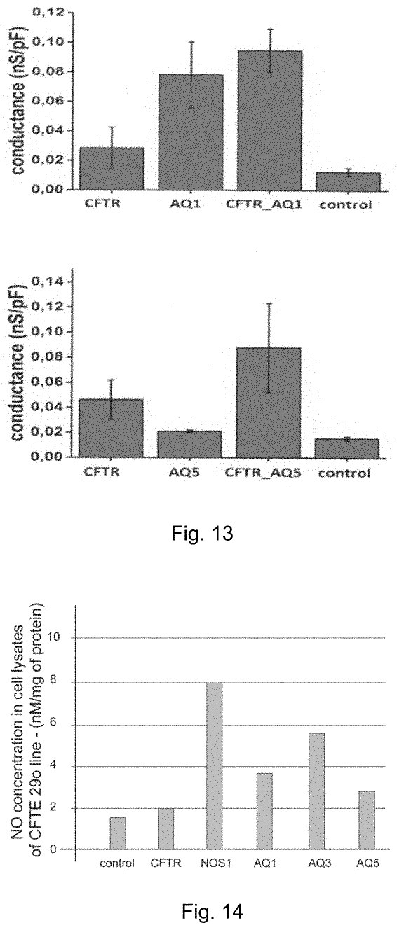

FIG. 13 shows the average conductivity of the cell membrane, normalised cell membranes to electrical capacitance (G/C=I/(UC) of CFTE29o—cells after their transfection with DNA vectors VTvaf17-CFTR, VTvaf17-AQ1, (expressing CFTR and AQ1 genes), as well as upon co-transfection of CFTE29o—cells with DNA vectors VTvaf17-CFTR and VTvaf17-AQ1 (A) and the average resting potential of CFTE29o—cells after their transfection with DNA vectors VTvaf17-CFTR, VTvaf17-AQ5 (expressing CFTR and AQ5 genes), as well as upon co-transfection of CFTE29o—cells with DNA vectors VTvaf17-CFTR and VTvaf17-AQ5 (B), where: control—cells transfected with DNA vector VTvaf17,

CFTR—CFTE29o—cells transfected with DNA vector VTvaf17-CFTR,
AQ1 —CFTE29o—cells transfected with DNA vector VTvaf17-AQ1,
AQ5 —CFTE29o—cells transfected with DNA vector VTvaf17-AQ5,
CFTR AQ1 —CFTE29o—cells co-transfected with DNA vectors VTvaf17-CFTR and VTvaf17-AQ1,
CFTR AQ5 —CFTE29o—cells co-transfected with DNA vectors VTvaf17-CFTR and VTvaf17-AQ5.

FIG. 14 shows the plot of nitric oxide concentration in cell lysates of CFTE29o—line after transfection and co-transfection of these cells with DNA vectors VTvaf17-CFTR, VTvaf17-NOS1, VTvaf17-AQ 1, VTvaf17-AQ3, VTvaf17-AQ5 each carrying the therapeutic gene, namely CFTR, NOS1, AQ1, AQ3, AQ5 gene, where:

control—CFTE29o cells transfected with DNA vector VTvaf17 with Jet-Pei transport system,
CFTR—CFTE29o cells transfected with DNA vector VTvaf17-CFTR with Jet-Pei transport system,
NOS1—CFTE29o cells transfected with DNA vector VTvaf17-NOS1 with Jet-Pei transport system,
AQ1—CFTE29o cells transfected with DNA vector VTvaf17-AQ1 with Jet-Pei transport system,
AQ3—CFTE29o cells transfected with DNA vector VTvaf17-AQ3 with Jet-Pei transport system,
AQ5—CFTE29o cells transfected with DNA vector VTvaf17-AQ5 with Jet-Pei transport system.

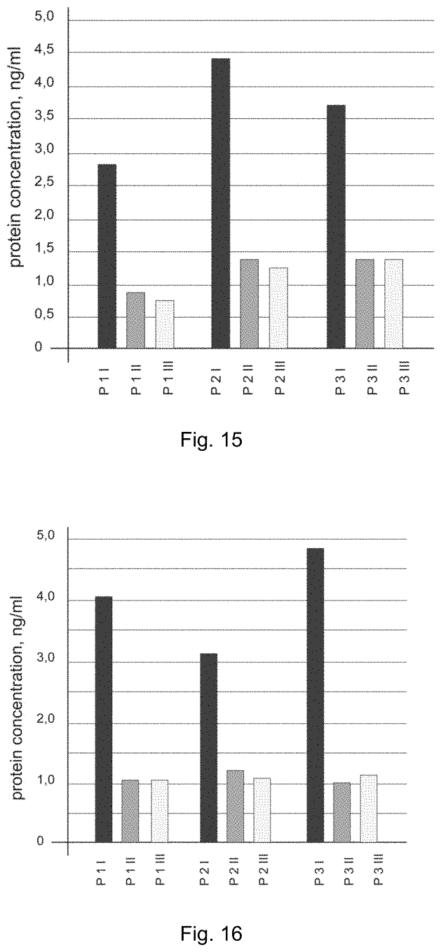

FIG. 15 shows the plot of aquaporin protein 1 concentration in skin biopsy specimens of three patients after the injection of the skin of these patients with gene therapy DNA vector VTvaf17-AQ1 carrying a therapeutic gene region, namely human AQ1 gene, for the purpose of analysing changes in aquaporin 1 protein concentration in human skin upon injection into human skin of gene therapy DNA vector VTvaf17-AQ1 carrying human AQ1 gene, where:

P1I—patient P1 skin biopsy in the region of injection of gene therapy DNA vector VTvaf17-AQ1 carrying a region of AQ1 gene,
P1II—patient P1 skin biopsy in the region of injection of gene therapy DNA vector VTvaf17 not carrying a region of AQ1 gene (placebo),
P1III—patient P1 skin biopsy from intact site,
P2I—patient P2 skin biopsy in the region of injection of gene therapy DNA vector VTvaf17-AQ1 carrying a region of AQ1 gene,
P2II—patient P2 skin biopsy in the region of injection of gene therapy DNA vector AQ1 not carrying a region of AQ1 gene (placebo),
P2III—patient P2 skin biopsy from intact site,
P3I—patient P3 skin biopsy in the region of injection of gene therapy DNA vector VTvaf17-AQ1 carrying a region of AQ1 gene,
P3II—patient P3 skin biopsy in the region of injection of gene therapy DNA vector VTvaf17 not carrying a region of AQ1 gene (placebo),
P3III—patient P3 skin biopsy from intact site.

FIG. 16 shows the plot of neuronal nitric oxide synthase 1 protein concentration in the gastrocnemius muscle biopsy specimens of three patients after injection of the gastrocnemius muscle of these patients with gene therapy DNA vector VTvaf17-NOS 1 carrying a region of therapeutic gene, namely human NOS 1 gene, for the purpose of analysing changes in neuronal nitric oxide synthase 1 protein concentration in the human gastrocnemius muscle upon injection of the human gastrocnemius muscle with gene therapy DNA vector VTvaf17-NOS 1 carrying the human NOS 1 gene, where:

P1I—patient P1 gastrocnemius muscle biopsy in the region of injection of gene therapy DNA vector VTvaf17-NOS1 carrying a region of NOS1 gene,
P1I—patient P1 gastrocnemius muscle biopsy in the region of injection of gene therapy DNA vector VTvaf17 not carrying a region of NOS1 gene (placebo),
P1III—patient P1 gastrocnemius muscle biopsy from intact site,
P2I—patient P2 gastrocnemius muscle biopsy in the region of injection of gene therapy DNA vector VTvaf17-NOS1 carrying a region of NOS1 gene,
P2II—patient P2 gastrocnemius muscle biopsy in the region of injection of gene therapy DNA vector NOS1 not carrying a region of NOS1 gene (placebo),
P2III—patient P2 gastrocnemius muscle biopsy from intact site,
P3I—patient P3 gastrocnemius muscle biopsy in the region of injection of gene therapy DNA vector VTvaf17-NOS1 carrying a region of NOS1 gene,
PI3I—patient P3 gastrocnemius muscle biopsy in the region of injection of gene therapy DNA vector VTvaf17 not carrying a region of NOS1 gene (placebo),
P3III—patient P3 gastrocnemius muscle biopsy from intact site.

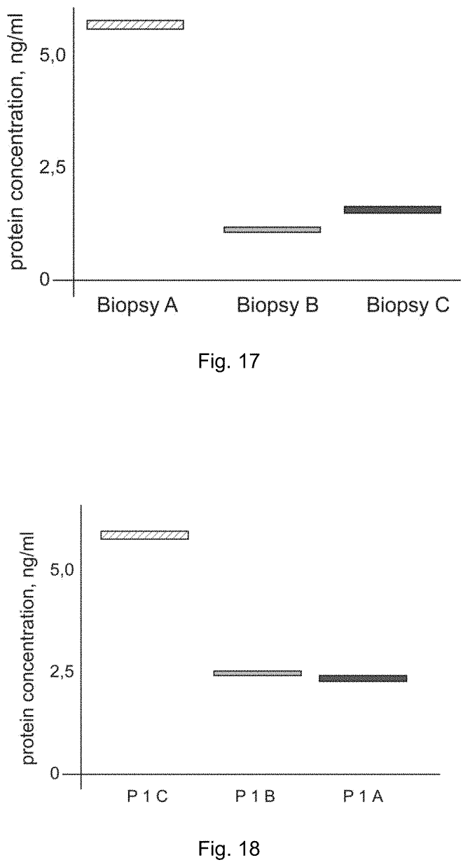

FIG. 17 shows the plot of protein concentration of transmembrane cystic fibrosis regulator protein in Sprague-Dawley rat lung and bronchial biopsy specimens after inhaled administration of gene therapy DNA vector VTvaf17-CFTR carrying a region of therapeutic gene, namely the human CFTR gene, into the cavity of lungs and bronchi of the rat for the purpose of analysing changes in transmembrane cystic fibrosis regulator protein concentration in the rat lungs and bronchi upon injection of the cavity of lungs and bronchi of the rat group with gene therapy DNA vector VTvaf17-CFTR carrying the human CFTR gene, where:

A—lung biopsy of the rat from control group (inhalation of physiological saline),
B—lung biopsy of the rat from control group (inhalation of DNA vector VTvaf17 not carrying cDNA of CFTR gene),
C—lung biopsy of the rat from control group (inhalation of DNA vector VTvaf17 carrying cDNA of CFTR gene).

FIG. 18 shows the plot of aquaporin 3 protein concentration in human skin biopsy specimens after the injection of the skin with culture of autologous fibroblasts transfected with gene therapy DNA vector VTvaf17-AQ3 carrying the therapeutic gene, namely the AQ3 gene, for the purpose of analysing changes in aquaporin 3 protein concentration in the skin compared to aquaporin 3 protein concentration in the patient's skin injection area of autologous fibroblasts non-transfected with gene therapy DNA vector VTvaf17-AQ3 carrying the therapeutic gene, namely the AQ3 gene (placebo), where:

P1A—patient P1 skin biopsy in the region of injection of autologous fibroblast culture of the patient transfected with gene therapy DNA vector VTvaf17-AQ3 carrying the AQ3 gene,
P1B—patient P1 skin biopsy in the region of injection of autologous fibroblast culture of the patient non-transfected with gene therapy DNA vector VTvaf17-AQ3 carrying the AQ3 gene,
P1C—patient P1 skin biopsy from intact skin site.

### EMBODIMENT OF THE INVENTION

Based on 3165 bp gene therapy DNA vector VTvaf17 (Cell and Gene Therapy LLC, PIT Ltd.), gene therapy DNA vectors carrying therapeutic human genes for the treatment of diseases associated with the need to increase the level of expression of these therapeutic genes have been constructed. A method for obtaining each gene therapy DNA vector carrying the therapeutic human genes is to clone the therapeutic gene to the polylinker of gene therapy DNA vector VTvaf17, namely CFTR (cystic fibrosis transmembrane conductance regulator gene or transmembrane cystic fibrosis regulator protein), NOS1 (neuronal nitric oxide synthase gene 1), AQ1 (aquaporin 1 gene), AQ3 (aquaporin 3 gene), AQ5 (aquaporin 5 gene).

Mutations and disruption of CFTR, NOS1, AQ1, AQ3, and AQ5 gene expression leads to the development of various human diseases.

The CFTR protein encoded by the cystic fibrosis transmembrane conductance regulator gene with the same name is a membrane channel for the active secretion of chloride ions from the cell into the lumen of excretory ducts of exocrine glands and acts as a switch regulating the absorption of sodium ions and participates in the regulation of other ion channels in the cell membrane. The gene mutation results in a disturbance of synthesis, structure, and function of said protein, resulting in impenetrability of chloride channels for chloride ions in case of hyperabsorption of sodium and simultaneous water entry into the cell. This leads to dehydration of the apical surface of secretory epithelium and therefore to an increase in the mucoviscosity. The disease caused by mutations in the CFTR gene is called cystic fibrosis. This is a hereditary multisystem disease affecting the respiratory airways, gastrointestinal tract, liver, pancreas, salivary glands, sweat glands, and reproductive system. At the same time, the respiratory tract pathology is the main cause of complications and mortality (National Consensus “Cystic Fibrosis: Definition, Diagnostic Criteria, Treatment” edited by E. I. Kondratieva, N. Yu. Kashirskaya, N. I. Kapranova. Moscow. 2016).

It was shown that the injection of a normal copy of the CFTR gene in a non-viral vector by nebulisation into the respiratory airways of patients with cystic fibrosis resulted in slight but pronounced therapeutic effect for 12 months compared to the control group (Alton E. et al. Repeated nebulisation of non-viral CFTR gene therapy in patients with cystic fibrosis: a randomised, double-blind, placebo-controlled, phase 2b trial. Lancet Respir Med. 2015 September; 3(9):684-691).

Protein of neuronal nitric oxide synthase 1 encoded by the NOS1 gene is an enzyme that catalyses the generation of nitric oxide and citrulline from arginine, oxygen, and NADPH. There are three NO-synthase isoforms in mammals: endothelial eNOS (or NOS3), neuronal nNOS (or NOS1), and inducible iNOS (or NOS2). The neuronal nitric oxide synthase 1 is expressed not only in the cells of nervous tissue, but also in muscles. The functions of neuronal nitric oxide are extremely varied: it controls the oscillatory neuronal activity, is a mediator of nociception, heat sensitivity, regulates the output of neuromediators. Nitric oxide produced in the brain is one of the most important levers for vascular tone control by the nervous system that delivers blood to all systems of the body.

Currently a whole group of diseases with expressed changes in the exchange of nitric oxide has been identified in research, as well as in clinical studies. First of all, these are cardiovascular diseases such as hypertension, myocardial infarction, atherosclerosis, and strokes. Changes in the activity of nitric oxide synthases are observed in diabetes, neurodegenerative and oncological diseases, acute and chronic inflammation of various organs and tissues. The extremely important role of nitric oxide in the regulation of vascular tone has been established. (E. V. Pozhilova, V. E. Novikov. Nitric Oxide Synthase and Endogenous Nitric Oxide in Cell Physiology and Pathology//Vestnik of the Smolensk State Medical Academy. 2015, No. 4, p. 35-41).

Recent studies point to a link between NOS1 gene activity and the severity of cystic fibrosis. It is shown that nitric oxide synthesised by NOS1 gene, the so-called “modifier gene”, affects the transepithelial ion transport, the immune response and development of nonspecific airway inflammation in cystic fibrosis (Texereau J, Marullo S, Hubert D, Coste J, Dusser DJ, Dall'Ava-Santucci J, Dinh-Xuan A T. Nitric oxide synthase 1 as a potential modifier gene of decline in lung function in patients with cystic fibrosis. Thorax. 2004. 59(2):156-158).

Aquaporins are a family of allosterically regulated proteins that form ion channels in the cytoplasmic membrane that allow water to penetrate. This family contains 11 members located in different organs. The space structure of these proteins resembles a cylindrical channel through which water molecules move, but not ions. Thanks to aquaporins, cells not only regulate their volume and internal pressure, but also perform such important functions as water absorption in the kidneys, etc. It is assumed that aquaporins are involved in the development of a number of hereditary and acquired diseases such as cerebral edema, cirrhosis, cardiac failure, glaucoma.

In addition, the close relationship of aquaporins 1, 3, and 5 with the cystic fibrosis transmembrane conductance regulator is shown. For instance, aquaporin 3 activates CFTR in epithelial cells of respiratory airways (Schreiber R, Nitschke R, Greger R, Kunzelmann K. The cystic fibrosis transmembrane conductance regulator activates aquaporin 3 in airway epithelial cells. J Biol Chem. 1999. 274(17):11811-11816.). AQ3 and AQ5 genes are involved in the regulation of fluid balance in epithelial cells in patients with cystic fibrosis (Levin M H, Sullivan S, Nielson D, Yang B, Finkbeiner W E, Verkman A S. Hypertonic saline therapy in cystic fibrosis: Evidence against the proposed mechanism involving aquaporins. J Biol Chem. 2006/281(35):25803-25812).

A method of producing a gene therapy DNA vector based on gene therapy DNA vector VTvaf17 carrying CFTR, or NOS1, or AQ1, or AQ3, or AQ5 therapeutic gene that involves obtaining each of the following group of gene therapy DNA vectors: DNA vector VTvaf17-CFTR, or VTvaf17-NOS1, or VTvaf17-AQ1, or VTvaf17-AQ3, or VTvaf17-AQ5 as follows: the coding region of CFTR, or NOS1, or AQ1, or AQ3, or AQ5 therapeutic gene is cloned to gene therapy DNA vector VTvaf17 and gene therapy DNA vector VTvaf17-CFTR, SEQ ID No. 1, or VTvaf17-NOS1, SEQ ID No. 2, or VTvaf17-AQ1, SEQ ID No. 3, or VTvaf17-AQ3, SEQ ID No. 4, or VTvaf17-AQ5, SEQ ID No. 5 is obtained, respectively, while the coding region of the therapeutic gene CFTR, or NOS1, or AQ1, or AQ3, or AQ5 is obtained by isolating the total RNA from a biological samples of human tissues followed by the reverse transcription reaction and PCR amplification using the produced oligonucleotides and cleavage of the amplification product by corresponding restriction endonucleases, and cloning to gene therapy DNA vector VTvaf17 performed by Sall and Kpnl restriction sites, or BamHI and EcoRI restriction sites, and the selection is made without antibiotics.

The coding region of CFTR gene (4459 bp), or NOS1 gene (4321 bp), or AQ1 gene (853 bp), or AQ3 gene (895 bp), or AQ5 gene (814 bp) was obtained by isolating total RNA from a biological sample of normal human tissue, followed by a reverse transcription reaction and PCR amplification using the oligonucleotides obtained by the chemical synthesis method for this purpose.

To synthesise the first strand of cDNA of human CFTR, NOS1, AQ1, AQ3, AQ5 genes, Mint reverse transcriptase (Evrogen, Russia) was used. 4 μl of Mint buffer, 2 μl of dithiothreitol, 2 μl of Mint reverse transcriptase, 2 μl of dNTP Mix and 2 μl of each of oligonucleotides obtained were added to 6 μl of total RNA:

for CFTR—CFTR_F and CFTR_R gene (list of oligonucleotide sequences (1) and (2)),
for NOS1 gene—NOS1_F and NOS1_R (list of oligonucleotide sequences (5) and (6)),
for AQ1 gene—AQ1_F and AQ1_R (list of oligonucleotide sequences (9) and (10)),
for AQ3 gene—AQ3_F and AQ3_R (list of oligonucleotide sequences (13) and (14)),
for AQ5 gene—AQ5_F and AQ5_R (list of oligonucleotide sequences (17) and (18)).

This mixture was incubated at 42° C. for 2 hours for each gene. The synthesised cDNA was used as a matrix in PCR amplification using the same oligonucleotides under the following conditions:

95° C.—1 min, 30 cycles: at 95° C. for 30 seconds, at 60° C. for 30 seconds and at 72° C. for 2 minutes, with final elongation at 72° C. for 3 minutes.

To confirm the efficiency of the obtained gene therapy DNA vector VTvaf17-CFTR carrying the therapeutic gene, namely the CFTR gene, gene therapy DNA vector VTvaf17-NOS1 carrying the therapeutic gene, namely the NOS1 gene, gene therapy DNA vector VTvaf17-AQ1 carrying the therapeutic gene, namely the AQ1 gene, gene therapy DNA vector VTvaf17-AQ3 carrying the therapeutic gene, namely the AQ3 gene, gene therapy DNA vector VTvaf17-AQ5 carrying the therapeutic gene, namely the AQ5 gene, the following was assessed:

A) change in mRNA accumulation of therapeutic genes in the cell lysate, after transfection of different cell lines with gene therapy DNA vectors,
B) change in the quantitative level of therapeutic proteins in the cell lysate after transfection of different cell lines with gene therapy DNA vectors,
C) change in the quantitative level of therapeutic proteins in the supernatant of human tissue biopsies after the injection of gene therapy DNA vectors into these tissues,
D) change in the quantitative level of therapeutic proteins in the supernatant of animal (rats) tissue biopsies after the injection of gene therapy DNA vectors into these tissues,
E) change in the quantitative level of therapeutic proteins in the supernatant of human tissue biopsies after the injection of autologous cells in these human tissues transfected with gene therapy DNA vectors,
F) change in the equilibrium potential of cells, the presence and level of chloride ion currents through the cell membrane after transfection of cell lines with gene therapy DNA vectors,
G) dependence of the ion current flowing through the cell membrane on the potential applied to it by measuring the conductance of cell membranes co-transfected with gene therapy DNA vectors,
H) change in the concentration of nitric oxide in cells after their transfection and co-transfection with gene therapy DNA vectors.

To confirm the practicability of the application of obtained gene therapy DNA vector VTvaf17-CFTR carrying the therapeutic gene, namely the CFTR gene, gene therapy DNA vector VTvaf17-NOS1 carrying the therapeutic gene, namely the NOS1 gene, gene therapy DNA vector VTvaf17-AQ1 carrying the therapeutic gene, namely the AQ1 gene, gene therapy DNA vector VTvaf17-AQ3 carrying the therapeutic gene, namely the AQ3 gene, gene therapy DNA vector VTvaf17-AQ5 carrying the therapeutic gene, namely the AQ5 gene, the following was performed:

I) transfection with gene therapy DNA vectors of different human cell lines,
J) injection of gene therapy DNA vectors into different human and animal tissues,
K) injection of autologous cells transfected with gene therapy DNA vectors into human tissues.

To confirm the producibility and constructability on an industrial scale of gene therapy DNA vector VTvaf17-CFTR carrying the therapeutic gene, namely the CFTR gene, gene therapy DNA vector VTvaf17-NOS1 carrying the therapeutic gene, namely the NOS1 gene, gene therapy DNA vector VTvaf17-AQ1 carrying the therapeutic gene, namely the AQ1 gene, gene therapy DNA vector VTvaf17-AQ3 carrying the therapeutic gene, namely the AQ3 gene, gene therapy DNA vector VTvaf17-AQ5 carrying the therapeutic gene, namely the AQ5 gene the following was performed:

L) fermentation on an industrial scale of Escherichia coli strain SCS110-AF/VTvaf17-CFTR, or Escherichia coli strain SCS110-AF/VTvaf17-NOS1, or Escherichia coli strain SCS110-AF/VTvaf17-AQ1, or Escherichia coli strain SCS110-AF/VTvaf17-AQ3, or Escherichia coli strain SCS110-AF/VTvaf17-AQ5, each containing gene therapy DNA vector VTvaf17 carrying a region of the therapeutic gene, namely CFTR, or NOS1, or AQ1, or AQ3, or AQ5.

### Example 1

Production of the gene therapy DNA vector VTvaf17-CFTR carrying a region of the therapeutic gene, namely the CFTR gene.

Gene therapy DNA vector VTvaf17-CFTR was constructed by cloning the coding region of the CFTR gene to the DNA vector VTvaf17 by SalI and KpnI restriction sites. The coding region of CFTR gene (4459 bp) was obtained by isolating total RNA from the biological human tissue sample followed by reverse transcription reaction and PCR amplification using obtained oligonucleotides and cleavage of the amplification product with appropriate restriction endonucleases. The following oligonucleotides were used as obtained for this purpose:

CFTR_F
ATCGTCGACCGCCATGCAGAGGTCGCCT,
CFTR_R
TGGTACCTTAAAGCCTTGTATCTTGCACCTC,

This resulted in a 7606 bp DNA vector VTvaf17-CFTR containing nucleotide sequence SEQ ID No. 1 carrying a region of the therapeutic gene, namely 4459 bp CFTR gene, allowing for antibiotic-free selection. Gene therapy DNA vector VTvaf17 was constructed by consolidating six fragments of DNA derived from different sources:

(a) the origin of replication was produced by PCR amplification of a region of commercially available plasmid pBR322 with a point mutation,
(b) EF1a promoter region was produced by PCR amplification of a site of human genomic DNA,
(c) hGH-TA transcription terminator was produced by PCR amplification of a site of human genomic DNA, (d) the RNA-OUT regulatory site of transposon Tn10 was synthesised from oligonucleotides,
(e) kanamycin resistance gene was produced by PCR amplification of a site of commercially available human plasmid pET-28,
(f) the polylinker was produced by annealing two synthetic oligonucleotides.

PCR amplification was performed using the commercially available kit Phusion® High-Fidelity DNA Polymerase (New England Biolabs) as per the manufacturer's instructions. The fragments have overlapping regions allowing for their consolidation with subsequent PCR amplification. Fragments (a) and (b) were consolidated using oligonucleotides Ori-F and EF1-R, and fragments (c), (d), and (e) were consolidated using oligonucleotides hGH-F and Kan-R. Afterwards, the produced fragments were consolidated by restriction with subsequent ligation by sites BamHI and NcoI. This resulted in a plasmid still devoid of the polylinker. To add it, the plasmid was cleaved by BamHI and EcoRI sites followed by ligation with fragment (f). Therefore, a 4182 bp vector was constructed carrying the kanamycin resistance gene flanked by SpeI restriction sites. Then this gene was cleaved by SpeI restriction sites and the remaining fragment was ligated to itself. This resulted in a 3165 bp gene therapy DNA vector VTvaf17 that is recombinant and allows for antibiotic-free selection.

### Example 2

Production of the gene therapy DNA vector VTvaf17-NOS1 carrying a region of the therapeutic gene, namely the NOS1 gene.

Gene therapy DNA vector VTvaf17-NOS1 was constructed by cloning the coding region of the NOS1 gene to the DNA vector VTvaf17 by SalI and KpnI restriction sites. The coding region of NOS1 gene (4321 bp) was obtained by isolating total RNA from the human tissue biopsy sample followed by reverse transcription reaction and PCR amplification using obtained oligonucleotides and cleavage of the amplification product with appropriate restriction endonucleases. The following oligonucleotides were used as obtained for this purpose:

NOS1_F
ATCGTCGACTACCATGGAGGATCACATG,
NOS1_R
CGGTACCTTAGGAGCTGAAAACCTCATC.

This resulted in a 7468 bp DNA vector VTvaf17-NOS1 containing nucleotide sequence SEQ ID No. 2 carrying a region of the therapeutic gene, namely 4321 bp NOS1 gene, allowing for antibiotic-free selection. Gene therapy DNA vector VTvaf17 was constructed as described in Example 1.

### Example 3

Production of the gene therapy DNA vector VTvaf17-AQ1 carrying a region of the therapeutic gene, namely the human AQ1 gene.

Gene therapy DNA vector VTvaf17-AQ1 was constructed by cloning the coding region of the AQ1 gene to the DNA vector VTvaf17 by BamHI and EcoRI restriction sites. The coding region of AQ1 gene (853 bp) was obtained by isolating total RNA from the human tissue biopsy sample followed by reverse transcription reaction and PCR amplification using obtained oligonucleotides and cleavage of the amplification product with appropriate restriction endonucleases. The following oligonucleotides were used as obtained for this purpose:

AQ1_F
TGGATCCAGCGGTCTCAGGCCAAG,
AQ1_R
CCAGAATTCTTCTATTTGGGCTTCATCTC.

This resulted in a 3982 bp gene therapy DNA vector VTvaf17-AQ1 containing nucleotide sequence SEQ ID No. 3 carrying a region of 853 bp AQ1 gene allowing for antibiotic-free selection. Gene therapy DNA vector VTvaf17 was constructed as described in Example 1.

### Example 4

Production of the gene therapy DNA vector VTvaf17-AQ3 carrying a region of the therapeutic gene, namely the AQ3 gene.

Gene therapy DNA vector VTvaf17-AQ3 was constructed by cloning the coding region of the AQ3 gene to the DNA vector VTvaf17 by BamHI and EcoRI restriction sites. The coding region of AQ3 gene (895 bp) was obtained by isolating total RNA from the human tissue biopsy sample followed by reverse transcription reaction and PCR amplification using obtained oligonucleotides and cleavage of the amplification product with appropriate restriction endonucleases. The following oligonucleotides were used as obtained for this purpose:

AQ3_F
TGGATCCCGCCATGGGTCGACAG,
AQ3_R
TCTGAATTCTCAGATCTGCTCCTTGTGCT.

This resulted in a 4024 bp DNA vector VTvaf17-AQ3 containing nucleotide sequence SEQ ID No. 4 carrying a region of the therapeutic gene, namely 895 bp AQ3 gene, allowing for antibiotic-free selection. Gene therapy DNA vector VTvaf17 was constructed as described in Example 1.

### Example 5

Production of the gene therapy DNA vector VTvaf17-AQ5 carrying a region of the therapeutic gene, namely the AQ5 gene.

Gene therapy DNA vector VTvaf17-AQ5 was constructed by cloning the coding region of the AQ5 gene to the DNA vector VTvaf17 by BamHI and EcoRI restriction sites. The coding region of AQ5 gene (814 bp) was obtained by isolating total RNA from the human tissue biopsy sample followed by reverse transcription reaction and PCR amplification using obtained oligonucleotides and cleavage of the amplification product with appropriate restriction endonucleases. The following oligonucleotides were used as obtained for this purpose:

AQ5_F
AGGATCCCACCATGAAGAAGGAGGTG,
AQ5_R
GGAATTCTCAGCGGGTGGTCAGCTCC.

This resulted in a 3943 bp DNA vector VTvaf17-AQ5 containing nucleotide sequence SEQ ID No. 5 carrying a region of the therapeutic gene, namely 814 bp AQ5 gene, allowing for antibiotic-free selection. Gene therapy DNA vector VTvaf17 was constructed as described in Example 1.

### Example 6

Proof of the efficiency of gene therapy DNA vector VTvaf17-CFTR carrying the therapeutic gene, namely the CFTR gene, and practicability of its use.

To confirm the efficiency of gene therapy DNA vector VTvaf17-CFTR carrying the therapeutic gene, namely the CFTR gene, and practicability of its use, changes in mRNA accumulation of the CFTR therapeutic gene in CFTE29o—cell line were assessed (Cell Culture Collection of Vertebrates, Institute of Cytology, Academy of Sciences) representing the immortalised cells of human tracheal epithelium, homozygous for ΔF508 mutation 48 hours after their transfection with the gene therapy DNA vector VTvaf17-CFTR carrying the human CFTR gene.

CFTE29o—human tracheal epithelial cell line was grown in Petri dishes at 37° C. in the presence of 5% CO2, in the DMEM medium with 10% fetal bovine serum and 10 μg/ml of gentamicin. To achieve 90% confluence, 24 hours before the transfection procedure, the cells were seeded into a 24-well plate in the quantity of 5×104 cells per well. Lipofectamine 3000 (ThermoFisher Scientific, USA) was used as a transfection reagent. The transfection with gene therapy DNA vector VTvaf17-CFTR expressing the human CFTR gene was performed as follows. In test tube 1, 1 μl of DNA vector VTvaf17-CFTR solution (concentration 500 ng/μl) and 411 of reagent P3000 was added to 25 μl of medium Opti-MEM (Gibco). The preparation was mixed by gentle shaking. In test tube 2, 1 μl of Lipofectamine 3000 solution was added to 25 μl of medium Opti-MEM (Gibco). The preparation was mixed by gentle shaking. The contents from test tube 1 were added to the contents of test tube 2, and the mixture was incubated at room temperature for 5 minutes. The resulting solution was added dropwise to the cells in the volume of 40 μl.

CFTE29o—human tracheal epithelial cell line transfected with gene therapy DNA vector VTvaf17 devoid of the inserted therapeutic gene was used as a reference. Reference vector VTvaf17 for transfection was prepared as described above.

Extraction of total RNA from the transfected cells was performed as follows. 1 ml of Trizol Reagent (ThermoFisher Scientific) was added to the well with cells, homogenised and heated for 5 minutes at 65° C. The sample was centrifuged at 14,000 g for 10 minutes and heated again for 10 minutes at 65° C. Then 200 μl of chloroform was added, and the mixture was gently stirred and centrifuged at 14,000 g for 10 minutes. Then the water phase was isolated and mixed with 1/10 of the volume of 3M sodium acetate, pH 5.2, and an equal volume of isopropyl alcohol. The sample was incubated at −20° C. for 10 minutes and then centrifuged at 14,000 g for 10 minutes. The precipitated RNA were rinsed in 1 ml of 70% ethyl alcohol, air-dried, and dissolved in 10 μl of RNase-free water. To measure the level of expression of CFTR gene mRNA after transfection, real-time PCR method (SYBR Green Real Time PCR) was used. For the amplification of human CFTR-specific cDNA, CFTR_SF and CFTR_SR oligonucleotides were used (see the list of sequences, (3), (4)). The length of the amplification product is 328 bp. Beta-2 microglobulin (B2M) was used as a reference gene.

PCR amplification was performed using QuantiTect SYBR Green RT-PCR Kit (Qiagen, USA) or another real-time PCR kit in 20 μl of the amplification mixture containing: 25 μl of QuantiTect SYBR Green RT-PCR Master Mix, 2.5 mM of magnesium chloride, 0.5 μM of each primer, and 5 μl of total RNA. For the reaction, CFX96 amplifier (Bio-Rad, USA) was used under the following conditions: 1 cycle of reverse transcription at 42° C. for 30 minutes, denaturation at 98° C. for 15 minutes, followed by 40 cycles comprising denaturation at 94° C. for 15 s, annealing of primers at 60° C. for 30 s and elongation at 72° C. for 120 s. Positive control included amplicons from PCR on matrices represented by plasmids in known concentrations containing cDNA sequences of CFTR and B2M genes. Negative control included deionised water. Real-time quantification of the PCR products, i.e. CFTR and B2M gene cDNAs obtained by amplification, was conducted using the Bio-Rad CFX Manager 2.1 software.

To confirm increased expression of the CFTR gene in CFTE29o—human tracheal epithelial cell line after the transfection of these cells with gene therapy DNA vector VTvaf17-CFTR carrying a region of the CFTR gene, FIG. 2 shows a diagram of accumulation of PCR products corresponding to the following:

1—cDNA of CFTR gene after transfection with gene therapy vector VTvaf17,
2—cDNA of CFTR gene after transfection with gene therapy vector VTvaf17-CFTR carrying a region of CFTR gene,
3—cDNA of B2M gene after transfection with gene therapy vector VTvaf17,
4—cDNA of B2M gene after transfection with gene therapy vector VTvaf17-CFTR carrying a region of CFTR gene.

The figure shows that the level of the specific mRNA of human CFTR gene has grown massively as a result of transfection of CFTE29o—human tracheal epithelial cell line with gene therapy DNA vector VTvaf17-CFTR, which indicates the efficiency of gene therapy DNA vector VTvaf17-CFTR and confirms the practicability of its use.

### Example 7

Proof of the efficiency of gene therapy DNA vector VTvaf17-NOS1 carrying the therapeutic gene, namely the NOS1 gene, and practicability of its use.

To confirm the efficiency of gene therapy DNA vector VTvaf17-NOS1 carrying the therapeutic gene, namely the NOS1 gene, and practicability of its use, changes in the mRNA accumulation of NOS1 therapeutic gene were assessed in SH-SY5Y human neuroblastoma cells (ATCC CRL-2266) 48 hours after their transfection with gene therapy DNA vector VTvaf17-NOS1 carrying a region of human NOS1 gene.

The human neuroblastoma cells were grown in a DMEM medium containing 4.5 g/l of glucose and 10% of fetal bovine serum (Gibco) at 37° C. in the presence of 5% CO2. To achieve 90% confluence, 24 hours before the transfection procedure, the cells were seeded into a 24-well plate in the quantity of 5×104 cells per well. Lipofectamine 3000 (ThermoFisher Scientific, USA) was used as a transfection reagent. The transfection with gene therapy DNA vector VTvaf17-NOS1 expressing the human NOS1 gene was performed according to the procedure described in Example 6. Extraction of total RNA from the transfected cells and synthesis of the first cDNA strand was performed according to the procedure described in Example 6. To measure the level of expression of NOS1 gene mRNA after transfection, real-time PCR method (SYBR Green Real Time PCR) was used. To amplify human NOS1-specific cDNA, NOS1_SF and NOS1_SR oligonucleotides were used (see the list of sequences, (7), (8)). The length of the amplification product is 818 bp. Beta-2 microglobulin (B2M) was used as a reference gene.

PCR amplification was performed using QuantiTect SYBR Green RT-PCR Kit (Qiagen, USA) or another real-time PCR kit in 20 μl of the amplification mixture containing: 25 μl of QuantiTect SYBR Green RT-PCR Master Mix, 2.5 mM of magnesium chloride, 0.5 μM of each primer, and 5 μl of total RNA. For the reaction, CFX96 amplifier (Bio-Rad, USA) was used under the following conditions: 1 cycle of reverse transcription at 42° C. for 30 minutes, denaturation at 98° C. for 15 minutes followed by 40 cycles comprising denaturation at 94° C. for 15 s, annealing of primers at 60° C. for 30 s and elongation at 72° C. for 120 s. Positive control included amplicons from PCR on matrices represented by plasmids in known concentrations containing cDNA sequences of NOS1 and B2M genes. Negative control included deionised water. Real-time quantification of the PCR products, i.e. NOS1 and B2M gene cDNAs obtained by amplification, was conducted using the Bio-Rad CFX Manager 2.1 software.

To confirm increased expression of the NOS1 gene in SH-SY5Y (ATCC CRL-2266) human neuroblastoma cells, after transfection of these cells with gene therapy DNA VTvaf17-NOS1 carrying a region of the NOS1 gene, FIG. 3 shows a diagram of accumulation of PCR products corresponding to the following:

1—cDNA of NOS1 gene after transfection with gene therapy vector VTvaf17,
2—cDNA of NOS1 gene after transfection with gene therapy vector VTvaf17-NOS1 carrying a region of NOS1 gene,
3—cDNA of B2M gene after transfection with gene therapy vector VTvaf17,
4—cDNA of B2M gene after transfection with gene therapy vector VTvaf17-NOS1 carrying a region of NOS1 gene.

The figure shows that the level of the specific mRNA of human NOS1 gene has grown massively as a result of transfection with gene therapy DNA vector VTvaf17-NOS1 carrying the therapeutic gene, namely human NOS1 gene, which indicates the efficiency of gene therapy DNA vector VTvaf17-NOS1 and confirms the practicability of its use.

### Example 8

Proof of the efficiency of gene therapy DNA vector VTvaf17-AQ1 carrying the therapeutic gene, namely the AQ1 gene, and practicability of its use.

To confirm the efficiency of gene therapy DNA vector VTvaf17-AQ1 carrying the therapeutic gene, namely the AQ1 gene, and practicability of its use, changes in mRNA accumulation of the AQ1 therapeutic gene in CFTE29o—human tracheal epithelial cell line were assessed 48 hours after their transfection with the gene therapy DNA vector VTvaf17-AQ1 carrying a region of human AQ1 gene.

CFTE29o—human tracheal epithelial cell line was grown as described in Example 6. To achieve 90% confluence, 24 hours before the transfection procedure, the cells were seeded into a 24-well plate in the quantity of 5×104 cells per well. Lipofectamine 3000 (ThermoFisher Scientific, USA) was used as a transfection reagent. The transfection with gene therapy DNA vector VTvaf17-AQ1 expressing the human AQ1 gene was performed according to the procedure described in Example 6. CFTE29o—cells transfected with gene therapy DNA vector VTvaf17 were used as a reference. Extraction of total RNA from the transfected cells and synthesis of the first cDNA strand was performed according to the procedure described in Example 6. To measure the level of expression of AQ1 gene mRNA after transfection, real-time PCR method (SYBR Green Real Time PCR) was used. For the amplification of human AQ1-specific cDNA, AQ1_F and AQ1_SR oligonucleotides were used (see the list of sequences, (11), (12)). The length of amplification product is 357 bp. Beta-2 microglobulin (B2M) was used as a reference gene.

PCR amplification was performed using QuantiTect SYBR Green RT-PCR Kit (Qiagen, USA) or another real-time PCR kit in 20 μl of the amplification mixture containing: 25 μl of QuantiTect SYBR Green RT-PCR Master Mix, 2.5 mM of magnesium chloride, 0.5 μM of each primer, and 5 μl of total RNA. For the reaction, CFX96 amplifier (Bio-Rad, USA) was used under the following conditions: 1 cycle of reverse transcription at 42° C. for 30 minutes, denaturation at 98° C. for 15 minutes followed by 40 cycles comprising denaturation at 94° C. for 15 s, annealing of primers at 60° C. for 30 s and elongation at 72° C. for 30 s. Positive control included amplicons from PCR on matrices represented by plasmids in known concentrations containing cDNA sequences of AQ1 and B2M genes. Negative control included deionised water. Real-time quantification of the PCR products, i.e. AQ1 and B2M gene cDNAs obtained by amplification, was conducted using the Bio-Rad CFX Manager 2.1 software.

To confirm increased expression of the AQ1 gene in CFTE29o—line human cells after transfection of these cells with gene therapy DNA vector VTvaf17-AQ1 carrying a region of the AQ1 gene, FIG. 4 shows a diagram of accumulation of PCR products corresponding to the following:

1—cDNA of AQ1 gene after transfection with gene therapy vector VTvaf17,
2—cDNA of AQ1 gene after transfection with gene therapy vector VTvaf17-AQ1 carrying a region of AQ1 gene,
3—cDNA of B2M gene after transfection with gene therapy vector VTvaf17,
4—cDNA of B2M gene after transfection with gene therapy vector VTvaf17-AQ1 carrying a region of AQ1 gene.

The figure shows that the level of the specific mRNA of human AQ1 gene has grown massively as a result of transfection of human CFTE29o—line tracheal epithelial cells with gene therapy DNA vector VTvaf17-AQ1 carrying the therapeutic gene, namely the human AQ1 gene, which indicates the efficiency of gene therapy DNA vector VTvaf17-AQ1 and confirms the practicability of its use.

### Example 9

Proof of the efficiency of gene therapy DNA vector VTvaf17-AQ3 carrying the therapeutic gene, namely the AQ3 gene, and practicability of its use.

To confirm the efficiency of gene therapy DNA vector VTvaf17-AQ3 carrying the therapeutic gene, namely the AQ3 gene, and practicability of its use, changes in mRNA accumulation of the AQ3 therapeutic gene in human primary small airway epithelial cells HSAECs (ATCC PCS-301-01) were assessed 48 hours after their transfection with the gene therapy DNA vector VTvaf17-AQ3 carrying a region of human AQ3 gene.

The human primary small airway epithelial cells were grown using the culture medium included in the Bronchial Epithelial Growth Kit (ATCC PCS-300-040) according to the manufacturer's instructions. To achieve 90% confluence, 24 hours before the transfection procedure, the cells were seeded into a 24-well plate in the quantity of 5×104 cells per well.

Lipofectamine 3000 (ThermoFisher Scientific, USA) was used as a transfection reagent. The transfection with gene therapy DNA vector VTvaf17-AQ3 expressing the human AQ3 gene was performed according to the procedure described in Example 6. Human small airway epithelial cells (HSAECs) transfected with gene therapy DNA vector VTvaf17 were used as a reference. Extraction of total RNA from the transfected cells and synthesis of the first cDNA strand was performed according to the procedure described in Example 6. To measure the level of expression of AQ3 gene mRNA after transfection, real-time PCR method (SYBR Green Real Time PCR) was used. For the amplification of human AQ3-specific cDNA, AQ3_SF and AQ3_SR oligonucleotides were used (see the list of sequences, (15), (16)). The length of amplification product is 350 bp. Beta-2 microglobulin (B2M) was used as a reference gene.

PCR amplification was performed using QuantiTect SYBR Green RT-PCR Kit (Qiagen, USA) or another real-time PCR kit in 20 μl of the amplification mixture containing: 25 μl of QuantiTect SYBR Green RT-PCR Master Mix, 2.5 mM of magnesium chloride, 0.5 μM of each primer, and 5 μl of total RNA. For the reaction, CFX96 amplifier (Bio-Rad, USA) was used under the following conditions: 1 cycle of reverse transcription at 42° C. for 30 minutes, denaturation at 98° C. for 15 minutes followed by 40 cycles comprising denaturation at 94° C. for 15 s, annealing of primers at 60° C. for 30 s and elongation at 72° C. for 30 s. Positive control included amplicons from PCR on matrices represented by plasmids in known concentrations containing cDNA sequences of AQ3 and B2M genes. Negative control included deionised water. Real-time quantification of the PCR products, i.e. AQ3 and B2M gene cDNAs obtained by amplification, was conducted using the Bio-Rad CFX Manager 2.1 software.

To confirm increased expression of the AQ3 gene in human small airway epithelial cells after transfection of these cells with gene therapy DNA vector VTvaf17-AQ3 carrying a region of AQ3 gene, FIG. 5 shows a diagram of accumulation of PCR products corresponding to the following:

1—cDNA of AQ3 gene after transfection with gene therapy vector VTvaf17,
2—cDNA of AQ3 gene after transfection with gene therapy vector VTvaf17-AQ3 carrying a region of AQ3 gene,
3—cDNA of B2M gene after transfection with gene therapy vector VTvaf17,
4—cDNA of B2M gene after transfection with gene therapy vector VTvaf17-AQ3 carrying a region of AQ3 gene.

The figure shows that the level of the specific mRNA of human AQ3 gene has grown massively as a result of transfection of human primary small airway epithelial cells with gene therapy DNA vector VTvaf17-AQ3 carrying the therapeutic gene, namely the human AQ3 gene, which indicates the efficiency of gene therapy DNA vector VTvaf17-AQ3 and confirms the practicability of its use.

### Example 10

Proof of the efficiency of gene therapy DNA vector VTvaf17-AQ5 carrying the therapeutic gene, namely the AQ5 gene, and practicability of its use.

To confirm the efficiency of gene therapy DNA vector VTvaf17-AQ5 carrying the therapeutic gene, namely the AQ5 gene, and practicability of its use, changes in mRNA accumulation ofthe AQ5 therapeutic gene in human primary bladder smooth muscle cells HBdSMC (ATCC PCS-420-012) were assessed 48 hours after their transfection with the gene therapy DNA vector VTvaf17-AQ5 carrying a region of human AQ5 gene.

Human primary bladder smooth muscle cells HBdSMC (ATCC PCS-420-012) were grown in medium included in the Vascular Smooth Muscle Cell Growth Kit (ATCC® PCS-100-042™) at 37° C. in the presence of 5% CO2. To achieve 90% confluence, 24 hours before the transfection procedure, the cells were seeded into a 24-well plate in the quantity of 5×104 cells per well. To achieve 90% confluence, 24 hours before the transfection procedure, the cells were seeded into a 24-well plate in the quantity of 5×104 cells per well. Lipofectamine 3000 (ThermoFisher Scientific, USA) was used as a transfection reagent. The transfection with gene therapy DNA vector VTvaf17-AQ5 expressing the human AQ5 gene was performed according to the procedure described in Example 6. HBdSMC cells transfected with gene therapy DNA vector VTvaf17 were used as a reference. Extraction of total RNA from the transfected cells and synthesis of the first cDNA strand was performed according to the procedure described in Example 6. To measure the level of expression of AQ5 gene mRNA after transfection, real-time PCR method (SYBR Green Real Time PCR) was used. For the amplification of human AQ5-specific cDNA, AQ5_SF and AQ5_SR oligonucleotides were used (see the list of sequences, (19), (20)). The length of amplification product is 477 bp. Beta-2 microglobulin (B2M) was used as a reference gene.

PCR amplification was performed using QuantiTect SYBR Green RT-PCR Kit (Qiagen, USA) or another real-time PCR kit in 20 μl of the amplification mixture containing: 25 μl of QuantiTect SYBR Green RT-PCR Master Mix, 2.5 mM of magnesium chloride, 0.5 μM of each primer, and 5 μl of total RNA. For the reaction, CFX96 amplifier (Bio-Rad, USA) was used under the following conditions: 1 cycle of reverse transcription at 42° C. for 30 minutes, denaturation at 98° C. for 15 minutes, followed by 40 cycles comprising denaturation at 94° C. for 15 s, annealing of primers at 60° C. for 30 s and elongation at 72° C. for 30 s. Positive control included amplicons from PCR on matrices represented by plasmids in known concentrations containing cDNA sequences of AQ5 and B2M genes. Negative control included deionised water. Real-time quantification of the PCR products, i.e. AQ5 and B2M gene cDNAs obtained by amplification, was conducted using the Bio-Rad CFX Manager 2.1 software.

To confirm increased expression of the AQ5 gene in HBdSMC cells after transfection of these cells with gene therapy DNA vector VTvaf17-AQ5 carrying a region of AQ5 gene, FIG. 6 shows a diagram of accumulation of PCR products corresponding to the following:

1—cDNA of AQ5 gene after transfection with gene therapy vector VTvaf17,
2—cDNA of AQ5 gene after transfection with gene therapy vector VTvaf17-AQ5 carrying a region of AQ5 gene,
3—cDNA of B2M gene after transfection with gene therapy vector VTvaf17,
4—cDNA of B2M gene after transfection with gene therapy vector VTvaf17-AQ5 carrying a region of AQ5 gene.

The figure shows that the level of the specific mRNA of human AQ5 gene has grown massively as a result of transfection of HBdSMC cells with gene therapy DNA vector VTvaf17-AQ5 carrying the therapeutic gene, namely human AQ5 gene, which indicates the efficiency of gene therapy DNA vector VTvaf17-AQ5 and confirms the practicability of its use.

### Example 11

Proof of the efficiency of gene therapy DNA vector VTvaf17-CFTR carrying the therapeutic gene, namely the CFTR gene, and practicability of its use.

To confirm the efficiency of gene therapy DNA vector VTvaf17-CFTR carrying the therapeutic gene, namely the CFTR gene, and practicability of its use, changes in transmembrane cystic fibrosis regulator protein concentration in the cell lysate of human CFTE29o—line tracheal epithelial cells were assessed 48 hours after their transfection with the gene therapy DNA vector VTvaf17-CFTR carrying a region of human CFTR gene.

To assess changes in transmembrane cystic fibrosis regulator protein concentration, CFTE29o—human tracheal epithelial cell line was used. CFTE29o—human tracheal epithelial cell line was grown as described in Example 6.

Dendrimers of the 6th generation SuperFect Transfection Reagent (Qiagen, Germany) were used as transport molecule, aqueous dendrimer solution without DNA vector (A) and DNA vector VTvaf17 devoid of cDNA of the CFTR gene (B) as a reference, and DNA vector VTvaf17-CFTR carrying a region of the human CFTR gene as transfected agents. The DNA-dendrimer complex was prepared according to the manufacturer's procedure (QIAGEN, SuperFect Transfection Reagent Handbook, 2002) with some modifications. For cell transfection in one well of a 24-well plate, antibiotic-free DMEM medium was added to 1 μg of DNA vector dissolved in TE buffer to a final volume of 60 μl, then 5 μl of SuperFect Transfection Reagent was added and gently mixed by pipetting five times. The complex was incubated at room temperature for 10-15 minutes. Then the culture medium was taken from the wells, the wells were rinsed with 1 ml of PBS buffer. 350 μl of DMEM complete medium containing 10 μg/ml of gentamicin was added to the resulting complex, mixed gently, and added to the cells. The cells were incubated with the complexes for 2-3 hours at 37° C. in the presence of 5% CO2.

The medium was then removed carefully, and the live cell array was rinsed with 1 ml of PBS buffer. Then, DMEM complete medium containing 10 μg/ml of gentamicin was added and incubated for 24-48 hours at 37° C. in the presence of 5% CO2.

After transfection, 0.1 ml of 1N HCl were added to 0.5 ml of the culture broth, mixed thoroughly, and incubated for 10 minutes at room temperature. Then, the mixture was neutralised by adding 0.1 ml of 1.2N NaOH/0.5M HEPES (pH 7-7.6) and stirred thoroughly. Supernatant was collected and used to assay the therapeutic protein. The product of the CFTR gene was assayed by enzyme-linked immunosorbent assay (ELISA) using the ELISA Kit for Cystic Fibrosis Transmembrane Conductance Regulator (CFTR) (SEC425Hu Cloud-Clone Corp., USA). The sensitivity is 0.059 ng/ml, measurement range—0.156-10 ng/ml.

The kit uses highly specific antibodies to transmembrane cystic fibrosis regulator protein adsorbed to microplate wells. 100 μl of each of the diluted reference samples and tested samples were added to the wells and incubated for 2 hours at 37° C. Then 100 μl of reagent A was added, the plate was covered with adhesive tape and incubated for 1 hour at 37° C. Then the wells were rinsed three times with 350 μl of wash buffer, and 100 μl of reagent B was added with subsequent incubation for 30 minutes at 37° C. After incubation, the wells were washed five times with 350 μl of wash buffer, 90 μl of substrate solution was added and incubated for 20-25 minutes at 37° C. The reaction was terminated by adding 500 μl of inhibitor removal buffer, and optical density was measured at 450 nm using ChemWell Automated EIA and Chemistry Analyser (Awareness Technology Inc., USA). To measure the numerical value of concentration, the calibration curve constructed using the reference samples from the kit with known concentrations of the transmembrane cystic fibrosis regulator protein was used. R-3.0.2 was used for the statistical treatment of the results and data visualization (https://www.r-project.org/).

It is shown that transfection of CFTE29o—human tracheal epithelial cell line with DNA vector carrying cDNA of the CFTR gene results in the increased concentration of transmembrane cystic fibrosis regulator protein in human tracheal epithelial cells, which indicates the efficiency of gene therapy DNA vector VTvaf17-CFTR and confirms the practicability of its use. The results are shown in FIG. 7.

### Example 12

Proof of the efficiency of gene therapy DNA vector VTvaf17-NOS1 carrying the therapeutic gene, namely the NOS1 gene, and practicability of its use.

To confirm the efficiency of gene therapy DNA vector VTvaf17-NOS1 carrying the therapeutic gene, namely the NOS1 gene, and practicability of its use, changes in protein concentration of neuronal nitric oxide synthase 1 in lysate of human neuroblastoma cells SH-SY5Y (ATCC CRL-2266) were assessed after their transfection with gene therapy DNA vector VTvaf17-NOS1 carrying a region of human NOS1 gene.

Human neuroblastoma cell culture SH-SY5Y (ATCC CRL-2266) was used to assess changes in protein concentration of neuronal nitric oxide synthase 1. Cells were grown as described in Example 7. Dendrimers of the 6th generation SuperFect Transfection Reagent (Qiagen, Germany) were used as transport molecule, aqueous dendrimer solution without DNA vector (A) and DNA vector VTvaf17 devoid of cDNA of the NOS1 gene (B) as a reference, and DNA vector VTvaf17-NOS1 carrying a region of the human NOS1 gene SEQ ID No: 2(C) as transfected agents. Preparation of the DNA dendrimer complex and transfection of SH-SY5Y cells were performed according to the procedure described in Example 11.

After transfection, cells were rinsed three times with PBS, and then 1 ml of PBS was added to the cells and the cells were subjected to freezing/thawing three times. Then the suspension was centrifuged for 15 minutes at 15,000 rpm, and supernatant was collected and used for the quantification and assay of the therapeutic protein.

The product of NOS1 gene was assayed by enzyme-linked immunosorbent assay (ELISA) using the ELISA Kit for Nitric Oxide Synthase 1, Neuronal (NOS1) (SEA815Hu, Cloud-Clone Corp., USA). The sensitivity is 0.065 ng/ml, measurement range—0.156-10 ng/ml.

Preparation of test and standard samples, measurement and processing of results were performed according to the procedure described in Example 11.

It is shown that transfection of human neuroblastoma cell culture SH-SY5Y with DNA vector carrying cDNA of NOS1 gene results in the increased protein quantity of neuronal nitric oxide synthase 1 in SH-SY5Y cells, which indicates the efficiency of gene therapy DNA vector VTvaf17-NOS1 and confirms the practicability of its use. The results are shown in FIG. 8.

### Example 13

Proof of the efficiency of gene therapy DNA vector VTvaf17-AQ1 carrying AQ1 therapeutic gene and practicability of its use.

To confirm the efficiency of gene therapy DNA vector VTvaf17-AQ1 carrying AQ1 therapeutic gene and practicability of its use, changes in aquaporin 1 protein concentration in the cell lysate of CFTE29o—human tracheal epithelial cell line were assessed 48 hours after transfection of these cells with the gene therapy DNA vector VTvaf17-AQ1 carrying human AQ1 gene.

CFTE29o—human tracheal epithelial cell line was used to assess changes in aquaporin 1 protein concentration. As described in Example 6, dendrimers of the 6th generation SuperFect Transfection Reagent (Qiagen, Germany) were used as transport molecule, aqueous dendrimer solution without DNA vector (A) and DNA vector VTvaf17 devoid of cDNA of AQ1 gene (B) as a reference, and DNA vector VTvaf17-AQ1 carrying a region of human AQ1 gene SEQ ID No: 3 (C) as transfected agents. The DNA-dendrimer complex was prepared and human tracheal epithelial cells were transfected according to the procedure described in Example 11.

After transfection, cells were rinsed three times with PBS, and then 1 ml of PBS was added to the cells and the cells were subjected to freezing/thawing three times. Then the suspension was centrifuged for 15 minutes at 15,000 rpm, and supernatant was collected and used for the quantification and assay of the therapeutic protein.

The product of cDNA of AQ1 gene was assayed by enzyme-linked immunosorbent assay (ELISA) using the ELISA Kit for Aquaporin 1, Colton Blood Group (AQP1) (SEA579Hu, Cloud-Clone Corp., USA). The sensitivity is 0.09 ng/ml, measurement range—0.25-16 ng/ml.

Preparation of test and standard samples, measurement and processing of results were performed as described in Example 11.

It is shown that transfection of CFTE29o—human tracheal epithelial cell line with DNA vector carrying cDNA of AQ1 gene results in the increased concentration of aquaporin 1 protein in CFTE29o—human tracheal epithelial cell line, which indicates the efficiency of gene therapy DNA vector VTvaf17-AQ1 and confirms the practicability of its use. The results are shown in FIG. 9.

### Example 14

Proof of the efficiency of gene therapy DNA vector VTvaf17-AQ5 carrying AQ5 therapeutic gene and practicability of its use.

To confirm the efficiency of gene therapy DNA vector VTvaf17-AQ5 carrying AQ5 therapeutic gene and practicability of its use, changes in aquaporin 5 protein concentration in the human primary bladder smooth muscle cell lysate HBdSMC (ATCC PCS-420-012) were assessed after transfection of these cells with the gene therapy DNA vector VTvaf17-AQ5 carrying a region of human AQ5 gene.

The human primary bladder smooth muscle cells HBdSMC (ATCC PCS-420-012) was used to assess changes in aquaporin 5 protein concentration. As described in Example 10, dendrimers of the 6th generation SuperFect Transfection Reagent (Qiagen, Germany) were used as transport molecule, aqueous dendrimer solution without DNA vector (A) and DNA vector VTvaf17 devoid of cDNA of AQ5 gene (B) as a reference, DNA vector VTvaf17-AQ5 carrying a region of human AQ5 gene SEQ ID No: 5 (C) as transfected agents. The DNA-dendrimer complex was prepared and human bladder cells were transfected according to the procedure described in Example 11.

After transfection, cells were rinsed three times with PBS, and then 1 ml of PBS was added to the cells and the cells were subjected to freezing/thawing three times. Then the suspension was centrifuged for 15 minutes at 15,000 rpm, and supernatant was collected and used for the quantification and assay of the therapeutic protein.

The product of cDNA of AQ5 gene was assayed by enzyme-linked immunosorbent assay (ELISA) using the ELISA Kit for Aquaporin 5 (AQP5) (SEA583Hu, Cloud-Clone Corp., USA). The sensitivity is 0.055 ng/ml, measurement range—0.156-10 ng/ml.

Preparation of test and standard samples, measurement and processing of results were performed as described in Example 11.

It is shown that transfection of human primary bladder smooth muscle cells with DNA vector carrying cDNA of AQ5 gene results in the increased concentration of aquaporin 5 protein in human bladder cells, which indicates the efficiency of gene therapy DNA vector VTvaf17-AQ5 and confirms the practicability of its use. The results are shown in FIG. 10.

### Example 15

Proof of the efficiency of gene therapy DNA vector VTvaf17-AQ3 carrying the therapeutic gene, namely the AQ3 gene, and practicability of its use.

To confirm the efficiency of gene therapy DNA vector VTvaf17-AQ3 carrying AQ3 therapeutic gene and practicability of its use, changes in aquaporin 3 protein concentration in the human small airway epithelial cell lysate HSAECs (ATCC PCS-301-01) were assessed after transfection of these cells with the gene therapy DNA vector VTvaf17-AQ3 carrying a region of human AQ3 gene.

The human small airway epithelial cells HSAECs (ATCC PCS-301-01) were used to assess changes in aquaporin 3 protein concentration. As described in Example 10, dendrimers of the 6th generation SuperFect Transfection Reagent (Qiagen, Germany) were used as transport molecule, aqueous dendrimer solution without DNA vector (A) and DNA vector VTvaf17 devoid of cDNA of AQ3 gene (B) as a reference, and DNA vector VTvaf17-AQ3 carrying a region of human AQ3 gene SEQ ID No: 4 (C) as transfected agents. The DNA-dendrimer complex was prepared and human small airway epithelial cells HSAECs (ATCC PCS-301-01) were transfected according to the procedure described in Example 11.

After transfection, cells were rinsed three times with PBS, then 1 ml of PBS was added to the cells, and cells were subjected to freezing/thawing three times. Then the suspension was centrifuged for 15 minutes at 15,000 rpm, and supernatant was collected and used for the quantification and assay of the therapeutic protein.

The product of cDNA of AQ3 gene was assayed by enzyme-linked immunosorbent assay (ELISA) using the ELISA Kit for Aquaporin 3, Gill Blood Group (AQP3) (SEA581Hu, Cloud-Clone Corp., USA). The sensitivity is 0.058 ng/ml, measurement range—0.156-10 ng/ml.

Preparation of test and standard samples, measurement and processing of results were performed as described in Example 11.

It is shown that transfection of human small airway epithelial cells HSAECs (ATCC PCS-301-01) with DNA vector carrying cDNA of AQ3 gene results in the increased concentration of aquaporin 3 protein in human small airway epithelial cells HSAECs (ATCC PCS-301-01), which indicates the efficiency of gene therapy DNA vector VTvaf17-AQ3 and confirms the practicability of its use. The results are shown in FIG. 11.

### Example 16

Proof of the efficiency of gene therapy DNA vector VTvaf17-CFTR carrying the therapeutic gene, namely the CFTR gene, and practicability of its use.

To confirm the efficiency of gene therapy DNA vector VTvaf17-CFTR carrying the therapeutic gene, namely CFTR gene, and practicability of its use, the change in the equilibrium potential of CFTE29o—cells transfected with DNA vector VTvaf17-CFTR expressing the CFTR gene were assessed.

A series of buffer solutions was prepared for this purpose.

External buffer A: 145 mM NaCl; 10 mM HEPES; 10 mM D-glucose, pH 7.4

Internal buffer A: 145 mM KCl; 10 mM HEPES, pH 7.2

External buffer B: 140 mM NMDG (N-methyl-d-glucamine), 140 mM HCl, 2 mM CaCl2, 1 mM MgCl2, 10 mM HEPES, pH 7.4

Internal buffer B: 140 mM NMDG, 40 mM HCl, 100 mM L-glutamic acid, 0.2 mM CaCl2, 2 MgCl2, 1 mM EGTA, 10 mM HEPES, pH 7.2.

One day before transfection, CFTE29o—cells were subcultured in 6-well plates at a ratio of 6.5×105 cells per well (90% confluent monolayer) in the volume of 2 ml. In test tube No. 1, 5 μg of VTvaf17-CFTR vector (VTvaf17 vector devoid of cDNA of CFRT therapeutic gene was used as a reference) and 10 μl of P3000 reagent were added to 125 μl of Opti-MEM medium (Gibco) and mixed by gentle pipetting. In test tube No. 2, 7.5 μl of Lipofectamine3000 Reagent was added to 125 μl of Opti-MEM medium (Gibco) and mixed by gentle shaking (without vortex). Then the contents from test tube No. 2 were added dropwise to the contents of test tube No. 1 and mixed by pipetting 2 times. The mixture was incubated at room temperature for 5-10 minutes, then added dropwise to the cells. After 18 hours the medium was replaced with the fresh one. Cells were incubated for 30 hours.

2 hours before the start of measurement, CFTE29o—cells were subcultured in Petri dishes in order to obtain single cells. For this purpose, the medium was aspirated, and HBSS cells were rinsed (Thermo scientific, USA). The cells were detached with 200 μl of trypsin-EDTA (Thermo scientific, USA), and then the trypsin was inactivated with DMEM medium (Invitrogen) with 10% fetal bovine serum (Gibco) and gentamicin (Gibco), diluting to 2 ml. Then the cells were subcultured in 35 mm Petri dishes with 1:10-1:20 dilution.

2 hours after subculture, when cell adhesion reached 80-90%, the cells were rinsed 3 times with external buffer A and then the medium was replaced with 2 ml of external buffer A.

Measurement of the equilibrium potential of a cell was performed using the standard patch clamp technique. For this purpose, an Ag-AgCl reference electrode was placed in electrolyte solution washing the cells attached to the bottom of the Petri dish, and an Ag-AgCl measuring electrode was placed inside a micropipette filled with internal buffer A. The ion composition of external buffer corresponded to the extracellular solution, and the ion composition of electrolyte inside the pipette corresponded to the intracellular composition. The patch-pipette tip radius was 0.3-1 microns. Then the test difference of potentials was applied between the electrodes and the ion current in the patch clamp mode was measured using the Axopatch 200B patch clamp amplifier (Molecular Devices, USA). The voltage supplied to electrodes and measured current were digitised and recorded on electronic media using the DigitData 1550 data acquisition card (Molecular Devices, USA). The patch pipette was smoothly brought to the cell surface using nanopositioners (Sensapex, Finland) until a tight electrical contact between the pipette tip and the cytoplasmic membrane of the cell was formed. For this purpose, a slight negative hydrostatic pressure was created in the pipette at the moment of its contact with the cell membrane. Measurements were carried out only in cases where the electrical contact resistance was more than 1 GOhm. After the contact establishment, the contribution of pipette capacitance to the measured current was corrected for, and membrane portion inside the pipette was destroyed by high hydrostatic pressure. The sharp increase in capacitative current indicated there was electrical access to the cell. Then, the cell membrane capacitance was corrected for and the voltage required to be applied to the cell in order to set the current flowing through its membrane to zero was measured. This voltage corresponded to the resting cell potential.

Because the CFTR protein is involved in the transport of chloride ions, external and internal buffers B with positively charged Na+ and K+ ions replaced by NMDG were used to register chloride currents through the cell membrane, and hydrochloric acid was used as a source of chloride ion (Ettorre, M., et al., Electrophysiological evaluation of cystic fibrosis conductance transmembrane regulator (CFTR) expression in human monocytes. Biochim Biophys Acta, 2014. 1840(10): p. 3088-95).

The equilibrium potentials of non transfected CFTE29o—cells (group 1) and cells transfected with VTvaf17-CFTR vector carrying cDNA of CFTR therapeutic gene (group 2) and transfected with VTvaf17 vector not carrying cDNA of CFTR therapeutic gene (group 3) were measured under normal ionic conditions (NaCl outside, KCl inside). The limited sampling of cells (5 from each group) shows that there is no statistically significant difference between the cells group 1 and cells group 3 (control groups) under these conditions, for example, the average resting potential was −20±6 mV. Resting potential for cells group 2 was −14±7 mV.

Similar experiments on measuring the resting potential of cells by replacing positively charged ions with NMDG were carried out, and hydrochloric acid was used as a source of chloride ion. In this case, cells from the group 1 and 3 had a resting potential equal to 0 mV, while the potential of group 2 was equal to −30 mV, which corresponds to the expected Nernst potential for chloride ions (140 mM outside and 40 mM inside the cell).

FIG. 12 shows the graph confirming that transfection of CFTE29o—cells with a vector expressing the CFTR gene results in chloride ion currents across the cellular membrane with no signs of them in the non-transfected cell line, which indicates the efficiency of the gene therapy DNA vector VTvaf17-CFTR and confirms the practicability of its use.

### Example 17

Proof of the efficiency of gene therapy DNA vectors VTvaf17-CFTR, VTvaf17-AQ1, and VTvaf17-AQ5 each carrying the therapeutic gene, namely CFTR, AQ1, and AQ5 gene, and practicability of their use.

To confirm the efficiency of gene therapy DNA vectors VTvaf17-CFTR, VTvaf17-AQ1, and VTvaf17-AQ5 each carrying the therapeutic gene, namely CFTR, AQ1, and AQ5 gene, and practicability of their use, the dependence of the ion current flowing through the cell membrane (CFTE29o—cell line) on the potential applied to it was assessed. The conductance of CFTE29o—cell membranes co-transfected with DNA vectors VTvaf17-CFTR and VTvaf17-AQ1 (expressing CFTR and AQ1 genes), VTvaf17-CFTR and VTvaf17-AQ5 (expressing CFTR and AQ5 genes), as well as control vector VTvaf17 not carrying cDNA of CFTR therapeutic gene was measured.

For this purpose, the equilibrium potentials was measured for non-transfected CFTE29o—cells (control group), CFTE29o—cells co-transfected with DNA vectors VTvaf17-CFTR and VTvaf17 (CFTR group), CFTE29o—cells co-transfected with DNA vectors VTvaf17-AQ1 and VTvaf17 (AQ1 group), CFTE29o—cells co-transfected with DNA vectors VTvaf17-AQ5 and VTvaf17 (AQ5 group), CFTE29o—cells co-transfected with DNA vectors VTvaf17-CFTR and VTvaf17-AQ1 (CFTR_AQ1 group), CFTE29o—cells co transfected with DNA vectors VTvaf17-CFTR and VTvaf17-AQ5 (CFTR_AQ5 group) in case of replacement of positively charged ions with NMDG, while hydrochloric acid was used as a source of chloride ions. Transfection of CFTE29o—cells and measurement of their current-voltage characteristics was performed as described in Example 16.

FIG. 13 shows a histogram of average cell membrane conductance values normalised to the electrical capacitance of cell membrane (G/C=I/(UC)) for four groups of cells.

It was shown that co transfection of CFTR genes and aquaporins (AQ1 or AQ5) results in amplification of chloride ion currents across the cell membrane of human tracheal epithelial CFTE29o—cells (cumulative effect), which indicates the efficiency of gene therapy DNA vectors VTvaf17-CFTR, VTvaf17-AQ1, and VTvaf17-AQ5 and confirms the practicability of their use. The highest efficiency is observed at co-transfection of cells with DNA vectors expressing the CFTR and AQ1 genes.

### Example 18

Proof of the efficiency of gene therapy DNA vectors VTvaf17-CFTR, VTvaf17-NOS1, VTvaf17-AQ1, VTvaf17-AQ3, and VTvaf17-AQ5 each carrying the therapeutic gene, namely CFTR, NOS1, AQ1, AQ3, and AQ5 genes, and practicability of their use.

To confirm the efficiency of gene therapy DNA vectors VTvaf17-CFTR, VTvaf17-NOS1, VTvaf17-AQ1, VTvaf17-AQ3, and VTvaf17-AQ5 each carrying the therapeutic gene, namely CFTR, NOS1, AQ1, AQ3, and AQ5 genes, and practicability of their use, the concentrations of nitric oxide in the cells of CFTE29o—line after transfection and co-transfection with DNA vectors containing CFTR, NOS1, AQ1, AQ3, and AQ5 genes were assessed.

24 hours before the transfection reaction, CFTE29o—line tracheal epithelial cells, homozygous for F508del mutation, were seeded in three 6-well plates at the concentration of 105 cells per well (the final volume of culture medium in the well was 4 ml).

Complete medium: DMEM/F12 (Gibco)+10% heat-inactivated FBS (Gibco)+1× Antibiotic-Antimycotic (Streptomycin-Penicillin (Gibco)).

Culture conditions: 37° C., in the presence of 5% CO2. Lipofectamine 3000 (Invitrogen) and JET-Pei (Polyplus transfection, France) were used as transport systems. DNA/transport system complexes were prepared as described in Example 6 and 11. Transfection was carried out as described in Example 6. 48 hours after transfection, cells of CFTE29o—line were detached from the support by intensive pipetting in 1×DPBS (Gibco) solution. The obtained cell-rich fluid was transferred to 2 ml Eppendorf tubes. The cells were pelleted for 10 minutes at 150 rcm, the supernatant was collected, and the precipitated cells were re-suspended in 250μl of deionised water.

To obtain a cell lysate, the cells were lysed in three freeze/thaw cycles: suspensions were frozen at −70° C. (for approximately 30 minutes), and then quickly thawed in the dry block heater at +37° C. and mixed on Vortex. The freezing-thawing procedure was repeated three times. Then the homogenate of lysed cells was pelleted at 13,000 rpm for 15 minutes. NO level was examined by enzyme-linked immunosorbent assay in obtained supernatants. The NO concentration in cell lysates was determined using ELISA test system (Biosource, MBS044841, 48T/Kit) according to the manufacturer's instructions. The sensitivity is 1 μM/l; concentration range—10-320 1 μM/l.

The obtained NO concentration values were normalised by protein level in cell lysates. The protein concentration in the samples was assessed by the Bradford protein assay. Coomassie Brilliant Blue R-250 was used as a dye (for 1 litre: 100 mg of Coomassie R-250 was dissolved in 50 ml of alcohol and 100 ml of orthophosphoric acid was added, made up to 1 litre with water and filtered through a paper filter). Before testing, cell lysates were 50-fold diluted. Serial dilutions of human serum albumin, 1 mg/ml, from 100 to 2.5 μg/ml were used for calibration curve tracing. Testing was carried out in a 96-1 plate, samples and calibration samples were introduced in the volume of 50 μl/l Bradford reagent—200 μl/l. Three technical replicates were made for each calibration point and each sample.

FIG. 14 shows the diagram of NO concentration in CFTE29o—cells after their transfection and co-transfection with gene therapy DNA vectors containing CFTR, NOS1, AQ1, AQ3, and AQ5 genes. The obtained results show that the JET-Pei transfection and co-transfection of CFTE29o—line tracheal epithelial cells with DNA vector containing NOS1 used as a delivery system results in the significant increase in the NO concentration in the cell lysates, which indicates the efficiency of the gene therapy DNA vector VTvaf17-NOS1. At the same time, the transfection of these cells with DNA vector containing the CFTR gene did not affect the NO level. A moderate increase in NO concentration was also detected in CFTE29o cells transfected with DNA vectors containing AQ1, AQ3, and AQ5, which also indicates the efficiency of the gene therapy DNA vectors VTvaf17-AQ1, VTvaf17-AQ3, and VTvaf17-AQ5. Example also confirms the practicability of gene therapy DNA vectors VTvaf17-NOS1, VTvaf17-AQ1, VTvaf17-AQ3, and VTvaf17-AQ5 use.

### Example 19

Proof of the efficiency of gene therapy DNA vector VTvaf17-AQ1 carrying the therapeutic gene, namely the AQ1 gene, and practicability of its use.

To confirm the efficiency of gene therapy DNA vector VTvaf17-AQ1 carrying the therapeutic gene, namely the AQ1 gene, and practicability of its use, changes in aquaporin 1 protein concentration in human skin upon injection of gene therapy DNA vector VTvaf17-AQ1 carrying the therapeutic gene, namely human AQ1 gene, were assessed.

To analyse changes in the concentration of aquaporin 1 protein, gene therapy DNA vector VTvaf17-AQ1 carrying a region of AQ1 gene with transport molecule was injected into the skin of three patients with concurrent injection of a placebo being gene therapy DNA vector VTvaf17 devoid of cDNA of AQ1 gene with transport molecule. Patient 1, man, 47 y.o. (P1); Patient 2, woman, 48 y.o. P2); Patient 3, man, 53 y.o. (P3). Polyethyleneimine Transfection reagent cGMP grade in-vivo-jetPEI (Polyplus Transfection, France-USA) was used as a transport system. Gene therapy DNA vector VTvaf17-AQ1 containing cDNA of AQ1 gene and gene therapy DNA vector VTvaf17 used as a placebo not containing cDNA of AQ1 gene were dissolved in sterile nuclease-free water. To obtain a gene construct, DNA-cGMP grade in-vivo-jetPEI complexes were prepared according to the manufacturer recommendations.

Gene therapy DNA vector VTvaf17 (placebo) and gene therapy DNA vector VTvaf17-AQ1 carrying a region of AQ1 gene were injected in the quantity of lmg for each genetic construct using the tunnel method with a 30G needle to the depth of 5 mm. The injectate volume of gene therapy DNA vector VTvaf17 (placebo) and gene therapy DNA vector VTvaf17-AQ1 carrying a region of AQ1 gene was 0.3 ml for each genetic construct. The points of injection of each genetic construct were located at 8 to 10 cm intervals.

The biopsy samples were taken on the 2nd day after the injection of the genetic constructs of gene therapy DNA vectors. The biopsy samples were taken from the patients' skin in the site of injection of gene therapy DNA vector VTvaf17-AQ1 carrying a region of AQ1 gene (I), gene therapy DNA vector VTvaf17 (placebo) (II), and from intact skin (III) using the skin biopsy device Epitheasy 3.5 (Medax SRL). The skin of patients in the biopsy site was preliminarily rinsed with sterile saline and anaesthetised with a lidocaine solution. The biopsy sample size was ca. 10 mm3, and the weight was approximately 11 mg. The sample was placed in a buffer solution containing 50 mM of Tris-HCl, pH 7.6, 100 mM of NaCl, 1 mM of EDTA, and 1 mM of phenylmethylsulfonyl fluoride, and homogenised to obtain a homogenised suspension. The suspension was then centrifuged for 10 minutes at 14,000 g. Supernatant was collected and used to assay the therapeutic protein.

The product of cDNA of AQ1 gene was assayed by enzyme-linked immunosorbent assay (ELISA) using the ELISA Kit for Aquaporin 1, Colton Blood Group (AQP1) (SEA579Hu, Cloud-Clone Corp., USA). The sensitivity is 0.09 ng/ml, measurement range—0.25-16 ng/ml. Preparation of test and standard samples, measurement and processing of results were performed as described in Example 11.

It was shown that the concentration of aquaporin 1 protein was increased in the skin of all three patients in the site of injection of gene therapy DNA vector VTvaf17-AQ1 carrying the therapeutic gene, namely human AQ1 gene, compared to the concentration of aquaporin 1 protein in the site of injection of gene therapy DNA vector VTvaf17 (placebo) devoid of region of human AQ1 gene, which indicates the efficiency of gene therapy DNA vector VTvaf17-AQ1 and confirms the practicability of its use.

The resulting values of concentration of aquaporin 1 protein in the skin of patients P1, P2, and P3 are shown in FIG. 15.

### Example 20

Proof of the efficiency of gene therapy DNA vector VTvaf17-NOS1 carrying the therapeutic gene, namely the NOS1 gene, and practicability of its use.

To confirm the efficiency of gene therapy DNA vector VTvaf17-NOS1 carrying the therapeutic gene, namely the NOS1 gene, and practicability of its use, changes in protein concentration of neuronal nitric oxide synthase 1 in the human gastrocnemius muscle upon injection of the human gastrocnemius muscle with gene therapy DNA vector VTvaf17-NOS1 carrying the therapeutic gene, namely the human NOS1 gene, were assessed.

To analyse changes in protein concentration of neuronal nitric oxide synthase 1, gene therapy DNA vector VTvaf17-NOS1 carrying a region of NOS1 gene with transport molecule was injected into gastrocnemius muscle of three patients with concurrent injection of a placebo being gene therapy DNA vector VTvaf17 devoid of cDNA of NOS1 gene with transport molecule. Patient 1, woman, 60 y.o. (P1); Patient 2, man, 42 y.o. (P2); Patient 3, man, 63 y.o. (P3). Polyethyleneimine Transfection reagent cGMP grade in-vivo-jetPEI (Polyplus Transfection, France-USA) was used as a transport system as described in Example 19.

Gene therapy DNA vector VTvaf17 (placebo) and gene therapy DNA vector VTvaf17-NOS1 carrying a region of NOS1 gene were injected in the quantity of 1 mg for each genetic construct using the tunnel method with a 30 G needle to the depth of 10 mm. The injectate volume of gene therapy DNA vector VTvaf17 (placebo) and gene therapy DNA vector VTvaf17-NOS1 carrying a region of NOS1 gene was 0.3 ml for each genetic construct. The points of injection of each genetic construct were located medially at 8 to 10 cm intervals.

The biopsy samples were taken on the 2nd day after the injection of the genetic constructs of gene therapy DNA vectors. The biopsy samples were taken from the patients' gastrocnemius muscle in the site of injection of gene therapy DNA vector VTvaf17-NOS1 carrying a region of NOS1 gene (I), gene therapy DNA vector VTvaf17 (placebo) (II), and intact skin (III) using the skin biopsy device MAGNUM (BARD, USA). The skin of patients in the biopsy site was preliminarily rinsed with sterile saline and anaesthetised with a lidocaine solution. The biopsy sample size was ca. 20 mm3, and the weight was up to 22 mg. The sample was placed in a buffer solution containing 50 mM of Tris-HCl, pH 7.6, 100 mM of NaCl, 1 mM of EDTA, and 1 mM of phenylmethylsulfonyl fluoride, and homogenised to obtain a homogenised suspension. The suspension was then centrifuged for 10 minutes at 14,000 g. Supernatant was collected and used to assay the therapeutic protein.

The product of cDNA of NOS1 gene was assayed by enzyme-linked immunosorbent assay (ELISA) using the ELISA Kit for Nitric Oxide Synthase 1, Neuronal (NOS1) (SEA815Hu, Cloud-Clone Corp., USA). The sensitivity is 0.065 ng/ml, measurement range—0.156-10 ng/ml. Preparation of test and standard samples, measurement and processing of results were performed as described in Example 11.

It was shown that in the gastrocnemius muscle of all three patients, the protein concentration of neuronal nitric oxide synthase 1 was increased in the site of injection of gene therapy DNA vector VTvaf17-NOS1 carrying the therapeutic gene, namely human NOS1 gene, compared to the protein concentration of neuronal nitric oxide synthase 1 in the site of injection of gene therapy DNA vector VTvaf17 (placebo) devoid of region of human NOS1 gene, which indicates the efficiency of gene therapy DNA vector VTvaf17-NOS1 and confirms the practicability of its use.

The results of measurement of protein concentration of neuronal nitric oxide synthase 1 in the gastrocnemius muscle biopsy specimens of patients P1, P2, P3 are shown in FIG. 16.

### Example 21

Proof of the efficiency of gene therapy DNA vector VTvaf17-CFTR carrying the therapeutic gene, namely the CFTR gene, and practicability of its use.

To confirm the efficiency of gene therapy DNA vector VTvaf17-CFTR carrying the therapeutic gene, namely the CFTR gene, and practicability of its use, changes in transmembrane cystic fibrosis regulator protein concentration in the rat lung and bronchial biopsy specimens upon injection of the cavity of lungs and bronchi of rats with gene therapy DNA vector VTvaf17-CFTR carrying a region of human CFTR gene were assessed.

In order to analyse the change in transmembrane cystic fibrosis regulator protein concentration, the gene therapy DNA vector VTvaf17-CFTR carrying a region of CFTR gene with a transport molecule was administered by inhalation into the cavity of lungs and bronchi often male Sprague-Dawley rats (group A). Gene therapy DNA vector VTvaf17 devoid of cDNA of CFTR gene with a transport molecule used as placebo was administered by inhalation to the control group of male Sprague-Dawley rats consisting of 5 animals (group B) into the cavity of lungs and bronchi. No external agents were administered into the cavity of lungs and bronchi of another control group of male Sprague-Dawley rats consisting of 5 animals (group C). Polyethyleneimine Transfection reagent cGMP grade in-vivo-jetPEI (Polyplus Transfection, France-USA) was used as a transport system as described in Example 19.

Gene therapy DNA vector VTvaf17 (placebo) and gene therapy DNA vector VTvaf17-CFTR carrying a region of CFTR gene were administered by inhalation into the cavity of lungs and bronchi in the quantity of 50 mg for each genetic construct. The injectate volume of gene therapy DNA vector VTvaf17 (placebo) and gene therapy DNA vector VTvaf17-CFTR carrying a region of CFTR gene was 0.3 ml for each genetic construct.

The biopsy samples were taken after necropsy of animals on the 3rd day after the injection of the gene therapy DNA vectors. Biopsies were taken from the bronchial and lung sites of rats of the target group upon administration of gene therapy DNA vector VTvaf17-CFTR carrying the CFTR gene (group A) and the control group upon administration of gene therapy DNA vector VTvaf17 (placebo) (group B) and intact animals (group C). The biopsy specimen size was ca. 10 mm3, and the weight was up to 12 mg. The sample was placed in a buffer solution containing 50 mM of Tris-HCl, pH 7.6, 100 mM of NaCl, 1 mM of EDTA, and 1 mM of phenylmethylsulfonyl fluoride, and homogenised to obtain a homogenised suspension. The suspension was then centrifuged for 10 minutes at 14,000 g. Supernatant was collected and used to assay the therapeutic protein.

The product of CFTR gene cDNA was assayed by enzyme-linked immunosorbent assay (ELISA) using the ELISA Kit for Cystic Fibrosis Transmembrane Conductance Regulator (CFTR) (SEC425Hu Cloud-Clone Corp., USA). The sensitivity is 0.059 ng/ml, measurement range—0.156-10 ng/ml.

Preparation of test and standard samples, measurement and processing of results were performed as described in Example 11.

It was shown that in bronchial and lung biopsy specimens of all animals of the target group, a significant amount of transmembrane cystic fibrosis regulator protein was recorded upon administration of gene therapy DNA vector VTvaf17-CFTR carrying the therapeutic gene, namely the human CFTR gene, compared to the amount of transmembrane cystic fibrosis regulator protein in bronchial and lung biopsy specimens of rats of the control group upon administration of gene therapy DNA vector VTvaf17 (placebo) devoid of region of human CFTR gene, which indicates the efficiency of gene therapy DNA vector VTvaf17-NOS1 and confirms the practicability of its use.

The results of measurement of protein concentration of transmembrane cystic fibrosis regulator protein in bronchial and lung biopsy specimens of rats are shown in FIG. 17.

### Example 22

Proof of the efficiency of gene therapy DNA vector VTvaf17-AQ3 carrying the therapeutic gene, namely the AQ3 gene, and practicability of its use.

To confirm the efficiency of gene therapy DNA vector VTvaf17-AQ3 carrying the therapeutic gene, namely the AQ3 gene, and practicability of its use, changes in concentration of aquaporin 3 protein in the human skin upon injection of the patient skin with autologous fibroblast culture transfected with the gene therapy DNA vector VTvaf17-AQ3 carrying the therapeutic gene, namely the AQ3 gene, were assessed. In order to analyse changes in the concentration of aquaporin 3 protein, the forearm skin of the patient was injected with the appropriate autologous fibroblast culture transfected with the gene therapy DNA vector VTvaf17-AQ3 carrying the therapeutic gene, namely the AQ3 gene with concurrent injection of a placebo in the form of autologous fibroblast culture non-transfected with gene therapy DNA vector VTvaf17-AQ3 carrying the therapeutic gene, namely the AQ3 gene.

The human primary culture of fibroblasts was grown from the patient skin biopsy specimens. Biopsy specimens of the skin from the area protected by ultraviolet, namely behind the ear or on the inner lateral side of the elbow, were taken using the skin biopsy device Epitheasy 3.5 (Medax SRL). The biopsy sample was ca. 10 mm and ca. 11 mg. The patient's skin was preliminarily rinsed with sterile saline and anaesthetised with a lidocaine solution. The primary cell culture was grown in Petri dishes at 37° C. in the presence of 5% CO2, in the DMEM medium with 10% fetal bovine serum and 100 U/ml of ampicillin. Trypsinisation and subculture was performed every 5 days. For trypsinisation, cells were rinsed with PBS buffer and incubated for 30 minutes at 37° C. in a solution containing 0.05% of trypsin and 1 mM of EDTA. For trypsin neutralisation, 5 ml of culture medium was added to the cells, and the suspension was centrifuged at 600 rpm for 5 minutes. After 4 or 5 passages, the fibroblast culture was cultivated in 75 ml culture flasks in DMEM medium containing 10 g/l glucose, 3.7 g/l of Na2CO3, 2.4 g/l of HEPES, 10% fetal serum, and 100 U/ml of ampicillin. The culture medium was replaced every 2 days. Total duration of culture growth did not exceed 25-30 days. Then an aliquot of 5×104 cells was taken from the cell culture. The patient-derived fibroblast culture was transfected with gene therapy DNA vector VTvaf17-AQ3 carrying the therapeutic gene, namely the AQ3 gene.

The transfection was carried out using a cationic polymer such as polyethyleneimine JETPEI (Polyplus transfection, France), according to the manufacturer's instructions. The cells were cultured for 72 hours and then injected into the patient. Injection of autologous fibroblast culture of the patient transfected with gene therapy DNA vector VTvaf17-AQ3 carrying the therapeutic gene, namely the AQ3 gene, and autologous fibroblast culture of the patient non-transfected with gene therapy DNA vector VTvaf17-AQ3 carrying the therapeutic gene, namely the AQ3 gene, as a placebo was made in the forearm using the tunnel method with a 13 mm long 30 G needle to the depth of 3 mm. The concentration of modified autologous fibroblasts in the injected suspension was approximately 5 million cells per 1 ml of suspension, the dose of injected cells did not exceed 15 million. The points of injection of autologous fibroblast culture transfected with gene therapy DNA vector VTvaf17-AQ3 carrying the therapeutic gene, namely AQ3 gene, and autologous fibroblast culture non-transfected with gene therapy DNA vector VTvaf17-AQ3 carrying the therapeutic gene, namely AQ3 gene, as a placebo were located at a distance of 8-10 cm from each other.

Biopsy samples were taken on the 4th day after the injection of autologous fibroblast culture transfected with the gene therapy DNA vector VTvaf17-AQ3 carrying the therapeutic gene, namely AQ3 gene, and placebo. Biopsy was taken from the patient's skin in the site of injection of autologous fibroblast culture transfected with gene therapy DNA vector VTvaf17-AQ3 carrying the therapeutic gene, namely AQ3 gene (I), autologous fibroblast culture non-transfected with gene therapy DNA vector VTvaf17-AQ3 carrying AQ3 therapeutic gene (placebo) (II), as well as from intact skin site (III) using the skin biopsy device Epitheasy 3.5 (Medax SRL). The skin of patients in the biopsy site was preliminarily rinsed with sterile saline and anaesthetised with a lidocaine solution. The biopsy sample size was ca. 10 mm3, and the weight was approximately 11 mg. The sample was placed in a buffer solution containing 50 mM of Tris-HCl, pH 7.6, 100 mM of NaCl, 1 mM of EDTA, and 1 mM of phenylmethylsulfonyl fluoride, and homogenised to obtain a homogenised suspension. The suspension was then centrifuged for 10 minutes at 14,000 g. Supernatant was collected and used to assay the therapeutic protein.

The product of AQ3 gene cDNA was assayed by enzyme-linked immunosorbent assay (ELISA) using the ELISA Kit for Aquaporin 3, Gill Blood Group (AQP3) (SEA581Hu, Cloud-Clone Corp., USA). The sensitivity is 0.058 ng/ml, measurement range—0.156-10 ng/ml. Preparation of test and standard samples, measurement and processing of results were performed as described in Example 11.

It was shown that in the patient's skin in the site of injection of autologous fibroblast culture transfected with gene therapy DNA vector VTvaf17-AQ3 carrying the therapeutic gene, namely AQ3 gene, the concentration of aquaporin 3 protein was increased compared to the concentration of aquaporin 3 protein in the patient's skin injection site of autologous fibroblast culture non-transfected with gene therapy DNA vector VTvaf17-AQ3 carrying the therapeutic gene, namely the AQ3 gene, (placebo), which indicates the efficiency of gene therapy DNA vector VTvaf17-AQ3 and confirms the practicability of its use. The results of measurement of aquaporin 3 protein concentrations in the patient's skin biopsy specimens are shown in FIG. 18.

### Example 23

Escherichia coli strain SCS110-AF/VTvaf17-CFTR, or Escherichia coli strain SCS110-AF/VTvaf17-NOS1, or Escherichia coli strain SCS110-AF/VTvaf17-AQ1, or Escherichia coli strain SCS110-AF/VTvaf17-AQ3, or Escherichia coli strain SCS110-AF/VTvaf17-AQ5 carrying gene therapy DNA vector, method of production thereof.

The strain construction for the production of gene therapy DNA vector based on gene therapy DNA vector VTvaf17 carrying CFTR, or NOS1, or AQ1, or AQ3, or AQ5 therapeutic gene on an industrial scale: namely Escherichia coli strain SCS110-AF/VTvaf17-CFTR, or Escherichia coli strain SCS110-AF/VTvaf17-NOS1, or Escherichia coli strain SCS110-AF/VTvaf17-AQ1, or Escherichia coli strain SCS110-AF/VTvaf17-AQ3, or Escherichia coli strain SCS110-AF/VTvaf17-AQ5 carrying gene therapy DNA vector VTvaf17-CFTR, or VTvaf17-NOS1, or VTvaf17-AQ1, or VTvaf17-AQ3, or VTvaf17-AQ5, respectively, for its production allowing for antibiotic-free selection involves making electrocompetent cells of Escherichia coli strain SCS110-AF and subjecting these cells to electroporation with gene therapy DNA vector VTvaf17-CFTR, or DNA vector VTvaf17-NOS1, or DNA vector VTvaf17-AQ1, or DNA vector VTvaf17-AQ3, or DNA vector VTvaf17-AQ5. After that, the cells were poured into agar plates (Petri dishes) with a selective medium containing yeastrel, peptone, 6% sucrose, and 10 μg/ml of chloramphenicol. At the same time, production of Escherichia coli strain SCS110-AF for the production of gene therapy DNA vector VTvaf17 or gene therapy DNA vectors based on it allowing for antibiotic-free positive selection involves constructing a 64 bp linear DNA fragment that contains regulatory element RNA-IN of transposon Tn10 allowing for antibiotic-free positive selection, a 1422 bp levansucrase gene sacB, the product of which ensures selection within a sucrose-containing medium, a 763 bp chloramphenicol resistance gene catR required for the selection of strain clones in which homologous recombination occurs, and two homologous sequences, 329 bp and 233 bp, ensuring homologous recombination in the region of gene recA concurrent with gene inactivation, and then the Escherichia coli cells are transformed by electroporation, and clones surviving in a medium containing 10 μg/ml of chloramphenicol are selected.

### Example 24

A method of production of gene therapy DNA vector based on gene therapy DNA vector VTvaf17 carrying CFTR, or NOS1, or AQ1, or AQ3, or AQ5 therapeutic gene.

To confirm the producibility and constructability on an industrial scale of gene therapy DNA vector VTvaf17-CFTR (SEQ ID [symbol] 1), or VTvaf17-NOS1 (SEQ ID [symbol] 2), or VTvaf17-AQ1 (SEQ ID [symbol] 3), or VTvaf17-AQ3 (SEQ ID [symbol] 4), or VTvaf17-AQ5 (SEQ ID [symbol] 5), each carrying a region of the therapeutic gene, namely CFTR, or NOS1, or AQ1, or AQ3, or AQ5, large-scale fermentation of Escherichia coli strain SCS110-AF/VTvaf17-CFTR (registered at the Russian National Collection of Industrial Microorganisms under number B-13169, INTERNATIONAL DEPOSITARY AUTHORITY No. NCIMB 43038), or Escherichia coli strain SCS110-AF/VTvaf17-NOS1 (registered at the Russian National Collection of Industrial Microorganisms under number B-13168, INTERNATIONAL DEPOSITARY AUTHORITY No. NCIMB 43037), or Escherichia coli strain SCS110-AF/VTvaf17-AQ1 (registered at the Russian National Collection of Industrial Microorganisms under number B-13172, INTERNATIONAL DEPOSITARY AUTHORITY No. NCIMB 43041), or Escherichia coli strain SCS110-AF/VTvaf17-AQ3 (registered at the Russian National Collection of Industrial Microorganisms under number B-13171, INTERNATIONAL DEPOSITARY AUTHORITY No. NCIMB 43040), or Escherichia coli strain SCS110-AF/VTvaf17-AQ5 (registered at the Russian National Collection of Industrial Microorganisms under number B-13170, INTERNATIONAL DEPOSITARY AUTHORITY No. NCIMB 43039) each containing gene therapy DNA vector VTvaf17 carrying a region of the therapeutic gene, namely CFTR, or NOS1, or AQ1, or AQ3, or AQ5, was performed. Each Escherichia coli strain SCS110-AF/VTvaf17-CFTR, or Escherichia coli strain SCS110-AF/VTvaf17-NOS1, or Escherichia coli strain SCS110-AF/VTvaf17-AQ1, or Escherichia coli strain SCS110-AF/VTvaf17-AQ3, or Escherichia coli strain SCS110-AF/VTvaf17-AQ5 was produced on the basis of Escherichia coli strain SCS110-AF (Cell and Gene Therapy LLC, PIT Ltd) as described in Example 23 by electroporation of competent cells of this strain with the gene therapy DNA vector VTvaf17-CFTR, or VTvaf17-NOS1, or VTvaf17-AQ1, or VTvaf17-AQ3, or VTvaf17-AQ5 carrying a region of the therapeutic gene, namely CFTR, or NOS1, or AQ1, or AQ3, or AQ3, with further inoculation of transformed cells into agar plates (Petri dishes) with a selective medium containing yeastrel, peptone, and 6% sucrose, and selection of individual clones.

Fermentation of Escherichia coli strain SCS110-AF/VTvaf17-CFTR carrying gene therapy DNA vector VTvaf17-CFTR was performed in a 101 fermenter with subsequent extraction of gene therapy DNA vector VTvaf17-CFTR.

For the fermentation of Escherichia coli strain SCS110-AF/VTvaf17-CFTR, medium containing the following ingredients per 101 of volume was prepared: 100 g of tryptone, 50 g of yeastrel (Becton Dickinson), then the medium was diluted with water to 8800 ml and autoclaved at 121° C. for 20 minutes, and then 1200 ml of 50% (w/v) sucrose was added. After that, the seed culture of Escherichia coli strain SCS110-AF/VTvaf17-CFTR was inoculated into a culture flask in the volume of 100 ml. The culture was incubated in an incubator shaker for 16 hours at 30° C. The seed culture was transferred to the Techfors S bioreactor (Infors HT, Switzerland) and grown to a stationary phase. The process was controlled by measuring optical density of the culture at 600 nm. The cells were pelleted for 30 minutes at 5,000-10,000 g. Supernatant was removed, and the cell pellet was re-suspended in 10% (by volume) phosphate buffered saline. The cells were centrifuged again for 30 minutes at 5,000-10,000 g. Supernatant was removed, a solution of 20 mM TrisCl, 1 mM EDTA, 200 g/l sucrose, pH 8.0 was added to the cell pellet in the volume of 1000 ml, and the mixture was stirred thoroughly to a homogenised suspension. Then egg lysozyme solution was added to the final concentration of 100 μg/ml. The mixture was incubated for 20 minutes on ice while stirring gently. Then 2500 ml of 0.2M NaOH, 10 g/l sodium dodecyl sulphate (SDS) was added, the mixture was incubated for 10 minutes on ice while stirring gently, then 3500 ml of 3M sodium acetate, 2M acetic acid, pH 5-5.5 was added, and the mixture was incubated for 10 minutes on ice while stirring gently. The resulting sample was centrifuged for 20-30 minutes at 15,000 g or a greater value. The solution was decanted delicately, and residual precipitate was removed by passing through a coarse filter (filter paper). Then RNase A (Sigma) was added to the final concentration of 20 μg/ml, and the solution was incubated overnight for 16 hours at room temperature. The solution was then centrifuged for 20-30 minutes at 15,000 g and passed through a 0.45 μm membrane filter (Millipore). Then ultrafiltration was performed with a membrane of 100 kDa (Millipore) and the mixture was diluted to the initial volume with a buffer solution of 25 mM TrisCl, pH 7.0. This manipulation was performed three to four times. The solution was applied to the column with 250 ml of DEAE Sepharose HP (GE, USA), equilibrated with 25 mM TrisCl, pH 7.0. After the application of the sample, the column was washed with three volumes of the same solution and then gene therapy DNA vector VTvaf17-CFTR was eluted using a linear gradient of 25 mM TrisCl, pH 7.0, to obtain a solution of 25 mM TrisCl, pH 7.0, 1 M NaCl, five times the volume of the column. The elution process was controlled by measuring optical density of the run-off solution at 260 nm. Chromatographic fractions containing gene therapy DNA vector VTvaf17-CFTR were joined together and subjected to gel filtration using Superdex 200 (GE, USA). The column was equilibrated with phosphate buffered saline. The elution process was controlled by measuring optical density of the run-off solution at 260 nm, and the fractions were analysed by agarose gel electrophoresis. The fractions containing gene therapy DNA vector VTvaf17-CFTR were joined together and stored at −20° C. To assess the process reproducibility, the indicated processing operations were repeated five times. All processing operations for Escherichia coli strain SCS110-AF/VTvaf17-NOS1, or Escherichia coli strain SCS110-AF/VTvaf17-AQ1, or Escherichia coli strain SCS110-AF/VTvaf17-AQ3, or Escherichia coli strain SCS110-AF/VTvaf17-AQ5 were performed in a similar way.

The process reproducibility and quantitative characteristics of final product yield confirm the producibility and constructability of gene therapy DNA vector VTvaf17-CFTR, or VTvaf17-NOS1, or VTvaf17-AQ1, or VTvaf17-AQ3, or VTvaf17-AQ5 on an industrial scale.

Therefore, the purpose of this invention, namely the construction of a gene therapy DNA vector carrying the therapeutic human genes based on gene therapy DNA vector VTvaf17 for the treatment of diseases associated with the need to increase the expression level of these therapeutic genes that would reasonably combine:

I) possibility of safe use in the gene therapy of human beings and animals due to the absence of antibiotic resistance genes in the gene therapy DNA vector,
II) length that ensures efficient gene delivery to the target cell,
III) presence of regulatory elements that ensure efficient expression of the therapeutic genes while not being represented by nucleotide sequences of viral genomes,
IV) producibility and constructability on an industrial scale, as well as the purpose of construction of strains carrying these gene therapy DNA vectors for the production of these gene therapy DNA vectors on an industrial scale has been achieved, which is supported by the following examples: for Item I—Example 1, 2, 3, 4, 5; 23 for Item II—Example 1, 2, 3, 4, 5, 6, 7, 8, 9, 10, 11, 12, 13, 14, 15,16, 17, 18, 19, 20; 21, 22; for Item III—Example 1, 2, 3, 4, 5, 6, 7, 8, 9, 10, 11, 12, 13, 14, 15,16, 17, 18, 19, 20; 21, 22; for Item IV—Example 23, 24.

### INDUSTRIAL APPLICABILITY

All the examples listed above confirm the industrial applicability of the proposed gene therapy DNA vector based on gene therapy DNA vector VTvaf17 carrying CFTR, or NOS1, or AQ1, or AQ3, or AQ5 therapeutic gene for the treatment of diseases associated with the need to increase the expression level of these therapeutic genes, method of its production and use, Escherichia coli strain SCS110-AF/VTvaf17-CFTR, or Escherichia coli strain SCS110-AF/VTvaf17-NOS1, or Escherichia coli strain SCS110-AF/VTvaf17-AQ1, or Escherichia coli strain SCS110-AF/VTvaf17-AQ3, or Escherichia coli strain SCS110-AF/VTvaf17-AQ5 carrying gene therapy DNA vector, method of its production, and the method of gene therapy DNA vector production on an industrial scale.

### LIST OF ABBREVIATIONS

VTvaf17—Gene therapy vector devoid of sequences of viral genomes and antibiotic resistance markers (vector therapeutic virus-antibiotic-free)

DNA—Deoxyribonucleic acid

cDNA—Complementary deoxyribonucleic acid

RNA—Ribonucleic acid

mRNA—Messenger ribonucleic acid

bp—base pair

PCR—Polymerase chain reaction

ml—millilitre, μl—microlitre

mm³—cubic millimetre

1—litre

μg—microgram

mg—milligram

g—gram

μM—micromol

mM—millimol

min—minute

s—second

rpm—rotations per minute

nm—nanometre

cm—centimetre

mW—milliwatt

RFU—Relative fluorescence unit

PBS—Phosphate buffered saline

What is claimed is:

1.-11. (canceled)

12. A gene therapy DNA vector based on a gene therapy DNA vector VTvaf17 carrying a therapeutic gene selected from a group of genes CFTR, NOS1, AQ1, AQ3, and AQ5 genes for a treatment of diseases featuring disruption of mucociliary transport and mucolytic function and development of mucostasis, including cystic fibrosis via an increase of expression of the selected therapeutic gene in humans and animals, while the gene therapy DNA vector has a coding region of the selected therapeutic gene cloned to the gene therapy DNA vector VTvaf17 resulting in the gene therapy DNA vector VTvaf17-CFTR that has nucleotide sequence SEQ ID No. 1, or VTvaf17-NOS1 that has nucleotide sequence SEQ ID No. 2, or VTvaf17-AQ1 that has nucleotide sequence SEQ ID No. 3, or VTvaf17-AQ3 that has nucleotide sequence SEQ ID No. 4, or VTvaf17-AQ5 that has nucleotide sequence SEQ ID No. 5, respectively, while each of the constructed gene therapy DNA vectors: VTvaf17-CFTR, or VTvaf17-NOS1, or VTvaf17-AQ1, or VTvaf17-AQ3, or VTvaf17-AQ5, due to a limited size of VTvaf17 vector part not exceeding 3200 bp, has an ability to effectively penetrate into human and animal cells and express the CFTR, or NOS1, or AQ1, or AQ3, or AQ5 therapeutic gene cloned to it and uses nucleotide sequences that are not antibiotic resistance genes, virus genes, or regulatory elements of viral genomes, which ensures its safe use for gene therapy in humans and animals.

13. A method of production of a gene therapy DNA vector based on a gene therapy DNA vector VTvaf17 carrying a therapeutic gene selected from the group of CFTR, NOS1, AQ1, AQ3, and AQ5 genes as per claim 12 that involves obtaining of each of the group of gene therapy DNA vectors: VTvaf17-CFTR, or VTvaf17-NOS1, or VTvaf17-AQ1, or VTvaf17-AQ3, or VTvaf17-AQ5 as follows: a coding region of the CFTR, or NOS1, or AQ1, or AQ3, or AQ5 therapeutic gene as per claim 12 is cloned into a gene therapy DNA vector VTvaf17, and the gene therapy DNA vector VTvaf17-CFTR, SEQ ID No. 1, or VTvaf17-NOS1, SEQ ID No. 2, or VTvaf17-AQ1, SEQ ID No. 3, or VTvaf17-AQ3, SEQ ID No. 4, or VTvaf17-AQ5, respectively, is obtained, while the coding region of the CFTR, or NOS1, or AQ1, or AQ3, or AQ5 therapeutic gene is obtained by isolating total RNA from a human biological tissue sample, followed by a reverse transcription reaction and a PCR amplification using the obtained oligonucleotides and cleaving an amplification product by corresponding restriction endonucleases, and cloning to the gene therapy DNA vector VTvaf17 is carried out at Nhel and Hindi!l restriction sites, while a selection is performed without antibiotics, while the method of production of gene therapy DNA vector allows for scaling a production volume of gene therapy DNA vector up to an industrial level.

14. Using of a gene therapy DNA vector based on a gene therapy DNA vector VTvaf17 carrying CFTR, or NOS1, or AQ1, or AQ3, or AQ5 therapeutic gene as described in claim 12 for a treatment of diseases featuring disruption of mucociliary transport and mucolytic function and development of mucostasis, including cystic fibrosis, via an increase of expression of CFTR, or NOS1, or AQ1, or AQ3, or AQ5 therapeutic genes in humans and animals that involves transfection of cells of human or animal organs and tissues with the selected gene therapy DNA vector carrying the therapeutic gene based on the gene therapy DNA vector VTvaf17 or several selected gene therapy DNA vectors carrying the therapeutic genes based on the gene therapy DNA vector VTvaf17 of the constructed gene therapy DNA vectors, and injection of human or animal autologous cells of said patient or animal transfected with the selected gene therapy DNA vector carrying the therapeutic gene or several selected gene therapy DNA vectors carrying the therapeutic genes of the constructed gene therapy DNA vectors carrying therapeutic genes into human or animal organs and tissues or injection of organs and tissues of the patient or animal with the selected gene therapy DNA vector or several selected gene therapy DNA vectors, or a combination of the indicated methods.


SEQUENCE LISTING (25 sequences)

SEQ ID NO: 1 - DNA, 7606 bp, Homo Sapiens
cgtgaggctccggtgcccgtcagtgggcagagcgcacatcgcccacagtccccgagaagttggggggaggggtcggcaattgaaccggtgcctagagaaagtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctaaaagccaggatccgatatcgtcgaccgccatgcagaggtcgcctctggaaaaggccagcgttgtctccaaactttttttcagctggaccagaccaattttgaggaaaggatacagacagcgcctggaattgtcagacatataccaaatcccttctgttgattctgctgacaatctatctgaaaaattggaaagagaatgggatagagagctggcttcaaagaaaaatcctaaactcattaatgcccttcggcgatgttttttctggagatttatgttctatggaatctttttatatttaggggaagtcaccaaagcagtacagcctctcttactgggaagaatcatagcttcctatgacccggataacaaggaggaacgctctatcgcgatttatctaggcataggcttatgccttctctttattgtgaggacactgctcctacacccagccatttttggccttcatcacattggaatgcagatgagaatagctatgtttagtttgatttataagaagactttaaagctgtcaagccgtgttctagataaaataagtattggacaacttgttagtctcctttccaacaacctgaacaaatttgatgaaggacttgcattggcacatttcgtgtggatcgctcctttgcaagtggcactcctcatggggctaatctgggagttgttacaggcgtctgccttctgtggacttggtttcctgatagtccttgccctttttcaggctgggctagggagaatgatgatgaagtacagagatcagagagctgggaagatcagtgaaagacttgtgattacctcagaaatgattgaaaatatccaatctgttaaggcatactgctgggaagaagcaatggaaaaaatgattgaaaacttaagacaaacagaactgaaactgactcggaaggcagcctatgtgagatacttcaatagctcagccttcttcttctcagggttctttgtggtgtttttatctgtgcttccctatgcactaatcaaaggaatcatcctccggaaaatattcaccaccatctcattctgcattgttctgcgcatggcggtcactcggcaatttccctgggctgtacaaacatggtatgactctcttggagcaataaacaaaatacaggatttcttacaaaagcaagaatataagacattggaatataacttaacgactacagaagtagtgatggagaatgtaacagccttctgggaggagggatttggggaattatttgagaaagcaaaacaaaacaataacaatagaaaaacttctaatggtgatgacagcctcttcttcagtaatttctcacttcttggtactcctgtcctgaaagatattaatttcaagatagaaagaggacagttgttggcggttgctggatccactggagcaggcaagacttcacttctaatggtgattatgggagaactggagccttcagagggtaaaattaagcacagtggaagaatttcattctgttctcagttttcctggattatgcctggcaccattaaagaaaatatcatctttggtgtttcctatgatgaatatagatacagaagcgtcatcaaagcatgccaactagaagaggacatctccaagtttgcagagaaagacaatatagttcttggagaaggtggaatcacactgagtggaggtcaacgagcaagaatttctttagcaagagcagtatacaaagatgctgatttgtatttattagactctccttttggatacctagatgttttaacagaaaaagaaatatttgaaagctgtgtctgtaaactgatggctaacaaaactaggattttggtcacttctaaaatggaacatttaaagaaagctgacaaaatattaattttgcatgaaggtagcagctatttttatgggacattttcagaactccaaaatctacagccagactttagctcaaaactcatgggatgtgattctttcgaccaatttagtgcagaaagaagaaattcaatcctaactgagaccttacaccgtttctcattagaaggagatgctcctgtctcctggacagaaacaaaaaaacaatcttttaaacagactggagagtttggggaaaaaaggaagaattctattctcaatccaatcaactctatacgaaaattttccattgtgcaaaagactcccttacaaatgaatggcatcgaagaggattctgatgagcctttagagagaaggctgtccttagtaccagattctgagcagggagaggcgatactgcctcgcatcagcgtgatcagcactggccccacgcttcaggcacgaaggaggcagtctgtcctgaacctgatgacacactcagttaaccaaggtcagaacattcaccgaaagacaacagcatccacacgaaaagtgtcactggcccctcaggcaaacttgactgaactggatatatattcaagaaggttatctcaagaaactggcttggaaataagtgaagaaattaacgaagaagacttaaaggagtgcttttttgatgatatggagagcataccagcagtgactacatggaacacataccttcgatatattactgtccacaagagcttaatttttgtgctaatttggtgcttagtaatttttctggcagaggtggctgcttctttggttgtgctgtggctccttggaaacactcctcttcaagacaaagggaatagtactcatagtagaaataacagctatgcagtgattatcaccagcaccagttcgtattatgtgttttacatttacgtgggagtagccgacactttgcttgctatgggattcttcagaggtctaccactggtgcatactctaatcacagtgtcgaaaattttacaccacaaaatgttacattctgttcttcaagcacctatgtcaaccctcaacacgttgaaagcaggtgggattcttaatagattctccaaagatatagcaattttggatgaccttctgcctcttaccatatttgacttcatccagttgttattaattgtgattggagctatagcagttgtcgcagttttacaaccctacatctttgttgcaacagtgccagtgatagtggcttttattatgttgagagcatatttcctccaaacctcacagcaactcaaacaactggaatctgaaggcaggagtccaattttcactcatcttgttacaagcttaaaaggactatggacacttcgtgccttcggacggcagccttactttgaaactctgttccacaaagctctgaatttacatactgccaactggttcttgtacctgtcaacactgcgctggttccaaatgagaatagaaatgatttttgtcatcttcttcattgctgttaccttcatttccattttaacaacaggagaaggagaaggaagagttggtattatcctgactttagccatgaatatcatgagtacattgcagtgggctgtaaactccagcatagatgtggatagcttgatgcgatctgtgagccgagtctttaagttcattgacatgccaacagaaggtaaacctaccaagtcaaccaaaccatacaagaatggccaactctcgaaagttatgattattgagaattcacacgtgaagaaagatgacatctggccctcagggggccaaatgactgtcaaagatctcacagcaaaatacacagaaggtggaaatgccatattagagaacatttccttctcaataagtcctggccagagggtgggcctcttgggaagaactggatcagggaagagtactttgttatcagcttttttgagactactgaacactgaaggagaaatccagatcgatggtgtgtcttgggattcaataactttgcaacagtggaggaaagcctttggagtgataccacagaaagtatttattttttctggaacatttagaaaaaacttggatccctatgaacagtggagtgatcaagaaatatggaaagttgcagatgaggttgggctcagatctgtgatagaacagtttcctgggaagcttgactttgtccttgtggatgggggctgtgtcctaagccatggccacaagcagttgatgtgcttggctagatctgttctcagtaaggcgaagatcttgctgcttgatgaacccagtgctcatttggatccagtaacataccaaataattagaagaactctaaaacaagcatttgctgattgcacagtaattctctgtgaacacaggatagaagcaatgctggaatgccaacaatttttggtcatagaagagaacaaagtgcggcagtacgattccatccagaaactgctgaacgagaggagcctcttccggcaagccatcagcccctccgacagggtgaagctctttccccaccggaactcaagcaagtgcaagtctaagccccagattgctgctctgaaagaggagacagaagaagaggtgcaagatacaaggctttaaggtaccgaattccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttacgcgtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaactagtccatggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcc

SEQ ID NO: 2 - DNA, 7468 bp, Homo Sapiens
cgtgaggctccggtgcccgtcagtgggcagagcgcacatcgcccacagtccccgagaagttggggggaggggtcggcaattgaaccggtgcctagagaaagtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctaaaagccaggatccgatatcgtcgactaccatggaggatcacatgttcggtgttcagcaaatccagcccaatgtcatttctgttcgtctcttcaagcgcaaagttgggggcctgggatttctggtgaaggagcgggtcagtaagccgcccgtgatcatctctgacctgattcgtgggggcgccgcagagcagagtggcctcatccaggccggagacatcattcttgcggtcaacggccggcccttggtggacctgagctatgacagcgccctggaggtactcagaggcattgcctctgagacccacgtggtcctcattctgaggggccctgaaggtttcaccacgcacctggagaccacctttacaggtgatgggacccccaagaccatccgggtgacacagcccctgggtccccccaccaaagccgtggatctgtcccaccagccaccggccggcaaagaacagcccctggcagtggatggggcctcgggtcccgggaatgggcctcagcatgcctacgatgatgggcaggaggctggctcactcccccatgccaacggcctggcccccaggcccccaggccaggaccccgcgaagaaagcaaccagagtcagcctccaaggcagaggggagaacaatgaactgctcaaggagatagagcctgtgctgagccttctcaccagtgggagcagaggggtcaagggaggggcacctgccaaggcagagatgaaagatatgggaatccaggtggacagagatttggacggcaagtcacacaaacctctgcccctcggcgtggagaacgaccgagtcttcaatgacctatgggggaagggcaatgtgcctgtcgtcctcaacaacccatattcagagaaggagcagccccccacctcaggaaaacagtcccccacaaagaatggcagcccctccaagtgtccacgcttcctcaaggtcaagaactgggagactgaggtggttctcactgacaccctccaccttaagagcacattggaaacgggatgcactgagtacatctgcatgggctccatcatgcatccttctcagcatgcaaggaggcctgaagacgtccgcacaaaaggacagctcttccctctcgccaaagagtttattgatcaatactattcatcaattaaaagatttggctccaaagcccacatggaaaggctggaagaggtgaacaaagagatcgacaccactagcacttaccagctcaaggacacagagctcatctatggggccaagcacgcctggcggaatgcctcgcgctgtgtgggcaggatccagtggtccaagctgcaggtattcgatgcccgtgactgcaccacggcccacgggatgttcaactacatctgtaaccatgtcaagtatgccaccaacaaagggaacctcaggtctgccatcaccatattcccccagaggacagacggcaagcacgacttccgagtctggaactcccagctcatccgctacgctggctacaagcagcctgacggctccaccctgggggacccagccaatgtgcagttcacagagatatgcatacagcagggctggaaaccgcctagaggccgcttcgatgtcctgccgctcctgcttcaggccaacggcaatgaccctgagctcttccagattcctccagagctggtgttggaagttcccatcaggcaccccaagtttgagtggttcaaggacctggggctgaagtggtacggcctccccgccgtgtccaacatgctcctagagattggcggcctggagttcagcgcctgtcccttcagtggctggtacatgggcacagagattggtgtccgcgactactgtgacaactcccgctacaatatcctggaggaagtggccaagaagatgaacttagacatgaggaagacgtcctccctgtggaaggaccaggcgctggtggagatcaatatcgcggttctctatagcttccagagtgacaaagtgaccattgttgaccatcactccgccaccgagtccttcattaagcacatggagaatgagtaccgctgccgggggggctgccctgccgactgggtgtggatcgtgccccccatgtccggaagcatcacccctgtgttccaccaggagatgctcaactaccggctcaccccctccttcgaataccagcctgatccctggaacacgcatgtctggaaaggcaccaacgggacccccacaaagcggcgagccatcggcttcaagaagctagcagaagctgtcaagttctcggccaagctgatggggcaggctatggccaagagggtgaaagcgaccatcctctatgccacagagacaggcaaatcgcaagcttatgccaagaccttgtgtgagatcttcaaacacgcctttgatgccaaggtgatgtccatggaagaatatgacattgtgcacctggaacatgaaactctggtccttgtggtcaccagcacctttggcaatggagatccccctgagaatggggagaaattcggctgtgctttgatggaaatgaggcaccccaactctgtgcaggaagaaaggaagagctacaaggtccgattcaacagcgtctcctcctactctgactcccaaaaatcatcaggcgatgggcccgacctcagagacaactttgagagtgctggacccctggccaatgtgaggttctcagtttttggcctcggctcacgagcataccctcacttttgcgccttcggacacgctgtggacaccctcctggaagaactgggaggggagaggatcctgaagatgagggaaggggatgagctctgtgggcaggaagaggctttcaggacctgggccaagaaggtcttcaaggcagcctgtgatgtcttctgtgtgggagatgatgtcaacattgaaaaggccaacaattccctcatcagcaatgatcgcagctggaagagaaacaagttccgcctcacctttgtggccgaagctccagaactcacacaaggtctatccaatgtccacaaaaagcgagtctcagctgcccggctccttagccgtcaaaacctccagagccctaaatccagtcggtcaactatcttcgtgcgtctccacaccaacgggagccaggagctgcagtaccagcctggggaccacctgggtgtcttccctggcaaccacgaggacctcgtgaatgccctgatcgagcggctggaggacgcgccgcctgtcaaccagatggtgaaagtggaactgctggaggagcggaacacggctttaggtgtcatcagtaactggacagacgagctccgcctcccgccctgcaccatcttccaggccttcaagtactacctggacatcaccacgccaccaacgcctctgcagctgcagcagtttgcctccctagctaccagcgagaaggagaagcagcgtctgctggtcctcagcaagggtttgcaggagtacgaggaatggaaatggggcaagaaccccaccatcgtggaggtgctggaggagttcccatctatccagatgccggccaccctgctcctgacccagctgtccctgctgcagccccgctactattccatcagctcctccccagacatgtaccctgatgaagtgcacctcactgtggccatcgtttcctaccgcactcgagatggagaaggaccaattcaccacggcgtatgctcctcctggctcaaccggatacaggctgacgaactggtcccctgtttcgtgagaggagcacccagcttccacctgccccggaacccccaagtcccctgcatcctcgttggaccaggcaccggcattgcccctttccgaagcttctggcaacagcggcaatttgatatccaacacaaaggaatgaacccctgccccatggtcctggtcttcgggtgccggcaatccaagatagatcatatctacagggaagagaccctgcaggccaagaacaagggggtcttcagagagctgtacacggcttactcccgggagccagacaaaccaaagaagtacgtgcaggacatcctgcaggagcagctggcggagtctgtgtaccgagccctgaaggagcaagggggccacatatacgtctgtggggacgtcaccatggctgctgatgtcctcaaagccatccagcgcatcatgacccagcaggggaagctctcggcagaggacgccggcgtattcatcagccggatgagggatgacaaccgataccatgaggatatttttggagtcaccctgcgaacgtacgaagtgaccaaccgccttagatctgagtccattgccttcattgaagagagcaaaaaagacaccgatgaggttttcagctcctaaggtaccgaattccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttacgcgtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaactagtccatggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcc

SEQ ID NO: 3 - DNA, 3982 bp, Homo Sapiens
cgtgaggctccggtgcccgtcagtgggcagagcgcacatcgcccacagtccccgagaagttggggggaggggtcggcaattgaaccggtgcctagagaaagtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctaaaagccaggatccagcggtctcaggccaagccccctgccagcatggccagcgagttcaagaagaagctcttctggagggcagtggtggccgagttcctggccacgaccctctttgtcttcatcagcatcggttctgccctgggcttcaaatacccggtggggaacaaccagacggcggtccaggacaacgtgaaggtgtcgctggccttcgggctgagcatcgccacgctggcgcagagtgtgggccacatcagcggcgcccacctcaacccggctgtcacactggggctgctgctcagctgccagatcagcatcttccgtgccctcatgtacatcatcgcccagtgcgtgggggccatcgtcgccaccgccatcctctcaggcatcacctcctccctgactgggaactcgcttggccgcaatgacctggctgatggtgtgaactcgggccagggcctgggcatcgagatcatcgggaccctccagctggtgctatgcgtgctggctactaccgaccggaggcgccgtgaccttggtggctcagccccccttgccatcggcctctctgtagcccttggacacctcctggctattgactacactggctgtgggattaaccctgctcggtcctttggctccgcggtgatcacacacaacttcagcaaccactggattttctgggtggggccattcatcgggggagccctggctgtactcatctacgacttcatcctggccccacgcagcagtgacctcacagaccgcgtgaaggtgtggaccagcggccaggtggaggagtatgacctggatgccgacgacatcaactccagggtggagatgaagcccaaatagaagaattccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttacgcgtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaactagtccatggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcc

SEQ ID NO: 4 - DNA, 4024 bp, Homo Sapiens
cgtgaggctccggtgcccgtcagtgggcagagcgcacatcgcccacagtccccgagaagttggggggaggggtcggcaattgaaccggtgcctagagaaagtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctaaaagccaggatcccgccatgggtcgacagaaggagctggtgtcccgctgcggggagatgctccacatccgctaccggctgctccgacaggcgctggccgagtgcctggggaccctcatcctggtgatgtttggctgtggctccgtggcccaggttgtgctcagccggggcacccacggtggtttcctcaccatcaacctggcctttggctttgctgtcactctgggcatcctcatcgctggccaggtctctggggcccacctgaaccctgccgtgacctttgccatgtgcttcctggctcgtgagccctggatcaagctgcccatctacaccctggcacagacgctgggagccttcttgggtgctggaatagtttttgggctgtattatgatgcaatctggcacttcgccgacaaccagctttttgtttcgggccccaatggcacagccggcatctttgctacctacccctctggacacttggatatgatcaatggcttctttgaccagttcataggcacagcctcccttatcgtgtgtgtgctggccattgttgacccctacaacaaccccgtcccccgaggcctggaggccttcaccgtgggcctggtggtcctggtcattggcacctccatgggcttcaactccggctatgccgtcaaccctgcccgggactttggcccccgcctttttacagcccttgcgggctggggctctgcagtcttcacgaccggccagcattggtggtgggtgcccatcgtgtccccactcctgggctccattgcgggtgtcttcgtgtaccagctgatgatcggctgccacctggagcagcccccaccctccaacgaggaagagaatgtgaagctggcccatgtgaagcacaaggagcagatctgagaattccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttacgcgtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaactagtccatggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcc

SEQ ID NO: 5 - DNA, 3943 bp, Homo Sapiens
cgtgaggctccggtgcccgtcagtgggcagagcgcacatcgcccacagtccccgagaagttggggggaggggtcggcaattgaaccggtgcctagagaaagtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctaaaagccaggatcccaccatgaagaaggaggtgtgctccgtggccttcctcaaggccgtgttcgcagagttcttggccaccctcatcttcgtcttctttggcctgggctcggccctcaagtggccgtcggcgctgcctaccatcctgcagatcgcgctggcgtttggcctggccataggcacgctggcccaggccctgggacccgtgagcggcggccacatcaaccccgccatcaccctggccctcttggtgggcaaccagatctcgctgctccgggctttcttctacgtggcggcccagctggtgggcgccattgccggggctggcatcctctacggtgtggcaccgctcaatgcccggggcaatctggccgtcaacgcgctcaacaacaacacaacgcagggccaggccatggtggtggagctgattctgaccttccagctggcactctgcatcttcgcctccactgactcccgccgcaccagccctgtgggctccccagccctgtccattggcctgtctgtcaccctgggccaccttgtcggaatctacttcactggctgctccatgaacccagcccgctcttttggccctgcggtggtcatgaatcggttcagccccgctcactgggttttctgggtagggcccatcgtgggggcggtcctggctgccatcctttacttctacctgctcttccccaactccctgagcctgagtgagcgtgtggccatcatcaaaggcacgtatgagcctgacgaggactgggaggagcagcgggaagagcggaagaagaccatggagctgaccacccgctgagaattccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttacgcgtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaactagtccatggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcc

SEQ ID NO: 6 - DNA, 28 bp, Artificial Sequence
  Oligonucleotide CFTR_F
atcgtcgaccgccatgcagaggtcgcct

SEQ ID NO: 7 - DNA, 31 bp, Artificial Sequence
  Oligonucleotide CFTR_R
tggtaccttaaagccttgtatcttgcacctc

SEQ ID NO: 8 - DNA, 18 bp, Artificial Sequence
  Oligonucleotide CFTR_SF
aggaggaacgctctatcg

SEQ ID NO: 9 - DNA, 18 bp, Artificial Sequence
  Oligonucleotide CFTR_SR
gcagacgcctgtaacaac

SEQ ID NO: 10 - DNA, 28 bp, Artificial Sequence
  Oligonucleotide NOS1_F
atcgtcgactaccatggaggatcacatg

SEQ ID NO: 11 - DNA, 28 bp, Artificial Sequence
  Oligonucleotide NOS1_R
cggtaccttaggagctgaaaacctcatc

SEQ ID NO: 12 - DNA, 20 bp, Artificial Sequence
  Oligonucleotide NOS1_SF
gacggcaagtcacacaaacc

SEQ ID NO: 13 - DNA, 20 bp, Artificial Sequence
  Oligonucleotide NOS1_SR
ggtttccagccctgctgtat

SEQ ID NO: 14 - DNA, 24 bp, Artificial Sequence
  Oligonucleotide AQ1_F
tggatccagcggtctcaggccaag

SEQ ID NO: 15 - DNA, 29 bp, Artificial Sequence
  Oligonucleotide AQ1_R
ccagaattcttctatttgggcttcatctc

SEQ ID NO: 16 - DNA, 20 bp, Artificial Sequence
  Oligonucleotide AQ1_SF
gctatgcgtgctggctacta

SEQ ID NO: 17 - DNA, 20 bp, Artificial Sequence
  Oligonucleotide AQ1_SR
ttgggcttcatctccaccct

SEQ ID NO: 18 - DNA, 23 bp, Artificial Sequence
  Oligonucleotide AQ3_F
tggatcccgccatgggtcgacag

SEQ ID NO: 19 - DNA, 29 bp, Artificial Sequence
  Oligonucleotide AQ3_R
tctgaattctcagatctgctccttgtgct

SEQ ID NO: 20 - DNA, 20 bp, Artificial Sequence
  Oligonucleotide AQ3_SF
acggtggtttcctcaccatc

SEQ ID NO: 21 - DNA, 20 bp, Artificial Sequence
  Oligonucleotide AQ3_SR
ggctgtgcctatgaactggt

SEQ ID NO: 22 - DNA, 26 bp, Artificial Sequence
  Oligonucleotide AQ5_F
aggatcccaccatgaagaaggaggtg

SEQ ID NO: 23 - DNA, 26 bp, Artificial Sequence
  Oligonucleotide AQ5_R
ggaattctcagcgggtggtcagctcc

SEQ ID NO: 24 - DNA, 20 bp, Artificial Sequence
  Oligonucleotide AQ5_SF
ctggcatcctctacggtgtg

SEQ ID NO: 25 - DNA, 20 bp, Artificial Sequence
  Oligonucleotide AQ5_SR
ccatggtcttcttccgctct